(19) United States
(12) Patent Application Publication
(10) Pub. No.: US 2026/0158115 A1
(43) Pub. Date: Jun. 11, 2026

(54) TYPE I COLLAGEN PEPTIDE FOR THERAPEUTIC USE

(71) Applicant: Gelita AG, Eberbach (DE)

(72) Inventors: Stephan Hausmanns, Heidelberg (DE); Hans-Ulrich Frech, Weinheim (DE); Steffen Oesser, Glücksburg (DE); Martin Hahn, Gronau (DE)

(21) Appl. No.: 18/723,198

(22) PCT Filed: Dec. 21, 2022
(86) PCT No.: PCT/EP2022/087199

§ 371 Date: Jun. 21, 2024

(30) Foreign Application Priority Data
Dec. 22, 2021 (DE) 10 2021 214 899.9

(51) Int. Cl. A61K 38/39; A61K 9/00; A61P 29/00; C07K 14/78

(52) U.S. Cl. CPC A61K 38/39; A61K 9/0053; A61P 29/00; C07K 14/78; C07K 2319/00

(57) ABSTRACT

The present invention relates to a recombinant, in particular non-denatured type I collagen for use in a therapeutic method for the oral therapy of inflammatory skin and mucous membrane diseases of a human or animal patient.

### CROSS REFERENCE TO RELATED APPLICATIONS

This application is a U.S. National Stage Application under 35 U.S.C. 371 of International Application No. PCT/EP2022/087199, filed Dec. 21, 2022, which claims priority to German Patent Application 10 2021 214 899.9, filed Dec. 22, 2021, the contents of each of which are hereby incorporated by reference in their entirety into the present disclosure.

### SEQUENCE LISTING

The contents of the electronic sequence listing (385721.xml; Size: 22,912 bytes; and Date of Creation: Jun. 21, 2024) is herein incorporated by reference in its entirety.

### DESCRIPTION

The present invention relates to a type I collagen, in particular a collagen peptide, for use in a therapeutic method for the oral therapy of inflammatory skin and mucous membrane diseases, in particular intestinal mucous membrane diseases, of a human or animal body.

Unfortunately, both the skin and the mucous membranes of humans and animals are frequently exposed to inflammatory diseases, and this with increasing tendency. In the course of intensive research into the aetiology of these diseases, it often turns out that such inflammatory diseases are caused by misdirected immune reactions, in particular autoimmune diseases. Examples of such inflammatory skin and mucous membrane diseases in the range of the skin are atopic dermatitis, neurodermatitis, psoriasis, rosacea and acne and in the range of the mucous membranes in particular intestinal mucous membrane inflammations such as Crohn's disease or ulcerative colitis.

A multiply used therapy is the intake of anti-inflammatory drugs and immunosuppressants such as cortisone, which suppress the body's own defences. However, suppressing the immune system's defence mechanism can also lead to many side effects, such as skin atrophy or pigmentation disorders as with cortisone-based topical therapy for atopic dermatitis. Other treatment options include the use of drugs that have a direct effect on the immune system, stem cell therapies, psychotherapy, occupational therapy, physiotherapy and nutritional counselling. Although therapeutically induced immunosuppression can lead to the protection of the affected organs, it can increase susceptibility to infection. Stem cell therapy is an ethically and legally controversial option for the treatment of autoimmune diseases and also harbours health risks.

It is known from U.S. Pat. Nos. 5,750,144, 5,645,851, 5,529,786 and 5,637,321 to provide animal tissue-containing compositions containing type II collagen for oral administration to treat an autoimmune disease, namely rheumatoid arthritis. In particular, these documents disclose the use of cartilage tissue-containing material recovered from natural animal sources to obtain a type II collagen-containing composition therefrom by means of various chemical-physical method steps. The type II collagen-containing compositions obtained in this way are characterised by the presence of non-denatured type II collagen on the one hand and a number of secondary components originating from the starting material and the isolation method, in particular proteins, antigens and salts, on the other. The therapy of inflammatory diseases of the skin and mucous membrane, in particular the intestinal mucous membrane, is not disclosed.

There is still a great need for therapeutic options, pharmaceutical compositions and active substances for the treatment of inflammatory skin and mucous membrane diseases, in particular intestinal mucous membrane diseases, in particular those that overcome the aforementioned problems. In particular, there is a great need for therapeutic options, pharmaceutical compositions and active substances which not only alleviate the suffering of the affected patients symptomatically, but causally treat, thus, eliminate the cause of the disease and/or its symptoms.

Therefore, the underlying technical problem of the present invention is to provide means, in particular active substances and compositions containing these with a therapeutic effect, as well as methods for the therapy of skin and mucous membrane inflammations, in particular skin and mucous membrane inflammations caused by autoimmune diseases, which overcome the aforementioned disadvantages, in particular which can be prepared in a standardised, reliable and precisely defined form, also on a larger industrial and cost-effective scale, and which are suitable for use in a method for the oral therapy of inflammatory skin and mucous membrane diseases of the human or animal body and/or for the preservation of skin and mucous membrane health due to their biological efficacy.

The present invention solves the technical problem underlying it by providing the teachings of the independent patent claims, in particular also the teachings of the preferred embodiments of the description and the dependent patent claims.

The present invention relates to a type I collagen for use in a therapeutic method for oral therapy of inflammatory skin and mucous membrane diseases of a human or animal patient, in particular immunomodulated inflammatory skin and mucous membrane diseases, in particular diseases of the intestinal mucous membrane, wherein the type I collagen has a molecular weight of at least 16 kDa.

The present invention relates in particular to a type I collagen for use in a therapeutic method for oral therapy of inflammatory skin and mucous membrane diseases, in particular intestinal mucosal diseases, of a human or animal patient, wherein the type I collagen has a molecular weight of at least 16 kDa and wherein the type I collagen is present in the form of a natural or recombinant type I collagen.

The surprising teaching underlying the present invention is that a recombinant or natural type I collagen, in particular in isolated form, is capable of treating inflammatory skin and mucous membrane diseases after oral application if this type I collagen has a molecular weight of at least 16 kDa. In a particularly preferred embodiment, the recombinant or natural type I collagens with a molecular weight of at least 16 kDa provided according to the invention are capable of treating inflammatory skin and mucous membrane diseases, in particular intestinal mucous membrane diseases, in particular those which are immunomodulated.

In a particularly preferred embodiment, recombinantly prepared type I collagens, in particular type I collagen peptides, are provided for this use according to the invention.

Surprisingly, the recombinant type I collagen provided according to the invention in a preferred embodiment, in particular recombinant type I collagen peptide, is capable of exhibiting a biological efficacy, in particular one that is at least the same as that which type I collagen recovered from natural sources according to the invention, i.e. natural type I collagen, in particular even an improved biological efficacy is provided. In a preferred embodiment, the present invention makes it possible to treat inflammatory skin and mucous membrane diseases in human or animal patients due to the high biological efficacy of the recombinant type I collagens, in particular type I collagen peptides, in very low dosages, i.e. low concentrations of the recombinant type I collagen, in particular type I collagen peptide. The teaching of the present invention therefore advantageously provides a reproducible, biotechnologically preparable recombinant type I collagen with a molecular weight of at least 16 kDa for the oral therapy of inflammatory skin and mucous membrane diseases, which can be prepared in a standardised manner, the preparation of which can be carried out on an industrial scale, and which can be prepared contamination-free in high purity and yield without the limitation of natural starting materials and is characterised in particular by the fact that it can be used in low doses due to its high biological efficacy.

The present invention also relates to type I collagen, in particular type I collagen peptides, with a molecular weight of at least 16 kDa for inducing oral tolerance, in particular for inducing oral tolerance to endogenously present collagen, in particular endogenously present type I collagen, in particular endogenous type I collagen present in skin or mucous membrane tissue, in particular in immunomodulated inflammatory skin and mucous membrane diseases, in particular intestinal mucous membrane diseases.

The present invention relates in particular to type I collagen, in particular recombinant type I collagen peptides, and compositions comprising type I collagen peptides, in particular recombinant type I collagen peptides, with a molecular weight of at least 16 kDa for use in a therapeutic method for the therapeutic treatment or therapeutic prophylaxis of immune intolerance to type I collagen, in particular by inducing immune tolerance to type I collagen, in particular in immunomodulated inflammatory skin and mucous membrane diseases, in particular intestinal mucosal diseases.

The present invention also relates to type I collagen, in particular recombinant type I collagen peptides, with a molecular weight of at least 16 kDa for use in a method for inducing immune tolerance to type I collagen, in particular a composition whose administration leads to inducing of oral tolerance to type I collagen, in particular in immunomodulated inflammatory skin and mucous membrane diseases, in particular intestinal mucous membrane diseases.

The natural and recombinant type I collagens used according to the invention, in particular recombinant type I collagen peptides, with a molecular weight of at least 16 kDa are characterised by a biological efficacy which develops after oral administration in human or animal bodies, in particular an immunomodulating and/or inflammation-modulating biological efficacy. In a particularly preferred embodiment, this biological efficacy is particularly present for natural and recombinant, preferably recombinant, type I collagens in non-denatured, i.e. native, form, and in a preferred embodiment also for recombinant type I collagen peptides.

Preferably, the type I collagens provided according to the invention, in particular type I collagen peptides, with a molecular weight of at least 16 kDa are capable of immunomodulation and/or inducing oral tolerance, in particular they effect an immune response and/or inducing oral tolerance in the treated human or animal body.

The natural or recombinant type I collagens with a molecular weight of at least 16 kDa provided according to the invention preferably unfold a biological efficacy suppressing the synthesis of immunoglobulins and/or an anti-inflammatory biological efficacy. According to the invention, the reduction of pro-inflammatory and the stimulation of anti-inflammatory cytokines could be determined. Without being bound by theory, it appears that the orally applied natural or recombinant type I collagen, in particular recombinant type I collagen peptide, provided according to the invention, with a molecular weight of at least 16 kDa, survives the gastrointestinal passage completely or largely undamaged and is recognised in immunomodulating, in particular immune suppressor cells, in particular cells of the Peyer's plaque, and triggers immunomodulating and/or cytokine-regulating reactions and/or signalling cascades which reduce or completely prevent undesirable immune reactions and inflammatory methods in the range of the skin and mucous membrane, in particular intestinal mucous membrane.

The oral application of natural or recombinant type I collagen, in particular type I collagen peptides, with a molecular weight of at least 16 kDa provided according to the invention appears, without being bound to theory, to induce an oral tolerance to endogenously present type I collagen triggering such undesirable reactions, so that a therapy of inflammatory skin and mucous membrane diseases, in particular intestinal mucous membrane diseases, is made possible.

The recombinant or natural type I collagens provided according to the invention, in particular recombinant type I collagen peptides, with a molecular weight of at least 16 kDa are preferably provided with the ability to interact with cells of the treated human or animal patient, in particular with cells of the Peyer's plaque, and in particular to lead to stimulation of anti-inflammatory and inhibition of pro-inflammatory cytokines as well as regeneration of immunosuppressive M2 macrophages and T-suppressor cells.

The inflammatory and immunomodulated skin and mucous membrane diseases that are particularly preferably treatable according to the invention are in particular diseases of the skin, such as cutaneous lupus erythematosus, dermatomyositis, lichen sclerosus, neurodermatitis, psoriasis, rosacea, lichen ruber, bullous pemphigoid, pemphigus vulgaris and acne or of the mucous membrane, in particular the intestinal mucous membrane, such as Crohn's disease, coeliac disease or ulcerative colitis.

In a particularly preferred embodiment, the present invention relates to a type I collagen, wherein the recombinant or natural collagen, in particular recombinant or natural type I collagen peptide is present in isolated, homogeneous form with a uniform molecular weight in a range of at least 16 kDa, in particular at least 20 kDa, or as an isolated collagen mixture, in particular collagen peptide mixture, wherein each collagen, in particular collagen peptide, has a molecular weight of at least 16 kDa, in particular at least 20 kDa.

The type 1 collagen used according to the invention, in particular recombinant or natural type I collagen, in particular type I collagen peptide, can therefore be present in one embodiment of the present invention in the form of a mixture with type 1 collagens, in particular type I collagen peptides, of exactly the same size and same molecular weight, i.e. in homogeneous form with a uniform molecular weight in a range of at least 16 kDa, in particular at least 20 kDa.

In a further embodiment of the present invention, the type I collagen used according to the invention, in particular recombinant or natural type 1 collagen, in particular type I collagen peptide, can be present in the form of a mixture with other type 1 collagens, in particular type I collagen peptides, which have a molecular weight differing from one another, wherein all type I collagens, in particular type I collagen peptides of the mixture, have a molecular weight of at least 16 kDa, in particular at least 20 kDa.

In a particularly preferred embodiment of the present invention, the recombinant or natural type I collagen used according to the invention, in particular the type I collagen peptide, with a molecular weight of at least 16 kDa is free of collagen, in particular also free of collagen peptides, with a molecular weight of less than 16 kDa.

In a particularly preferred embodiment of the present invention, the recombinant or natural type I collagen used according to the invention, in particular the type I collagen peptide, with a molecular weight of at least 16 kDa is free of type I collagen, in particular also free of type I collagen peptides, with a molecular weight of less than 16 kDa.

In a further particularly preferred embodiment, the recombinant or natural type I collagen can be present in the form of type I procollagen or mature type I collagen.

In a further particularly preferred embodiment, the recombinant or natural type I collagen can be present in triple-helical form, in particular in the form of a heterotrimer of two type I-α1 chains and one type I-α2 chain.

In a particularly preferred embodiment, the recombinant or natural type I collagen is present in non-denatured form, also referred to herein as native form, i.e. it has the naturally occurring tertiary and quaternary protein structure.

In a preferred embodiment, the present invention provides a type I collagen which can be present in the form of a type I collagen peptide, i.e. in single-stranded form, or which can be present in multi-stranded, for example double- or triple-stranded form, also referred to herein as triple-helical form, in particular in the form of a type I procollagen or mature type I collagen, in particular in the form of a heterotrimer of type I-α1 and type I-α2 chains.

Insofar as the natural or recombinant type I collagen according to the invention is not present as a single-stranded type I collagen peptide, but is present in, for example, triple-helical form, one or all of the individual collagen peptides that make up the triple-helical form of the type I collagen can be realized according to the present invention. In particular, the embodiments disclosed in the present teaching with respect to recombinant or natural type I collagen peptides also apply to type I collagens which have one, two or three such single-stranded type I collagen peptides, in particular are composed entirely of these, in particular consist of the natural, preferably recombinant, type I collagen peptides according to the invention.

In a particularly preferred embodiment, the recombinant or natural type I collagen can be bovine type I collagen.

In a particularly preferred embodiment, the recombinant or natural type I collagen can be type I-α1 or type I-α2 collagen, in particular type I-α1.

In a particularly preferred embodiment, the recombinant or recombinant type I collagen can be present in the form of cross-linked or non-cross-linked fibrils.

In a particularly preferred embodiment, the recombinant or natural type I collagen, in particular type I collagen peptide, can be a full-length collagen peptide, i.e. have the complete amino acid sequence of a naturally occurring collagen peptide of the type I.

In a further particularly preferred embodiment, the recombinant type I collagen is present in the form of a recombinant type I collagen peptide, in particular of type I-α1.

In a particularly preferred embodiment, the recombinant type I collagen, in particular type I collagen peptide, can be a type I collagen peptide with a molecular weight in a range of 16 to 400 Da, in particular 16 to 390 kDa, in particular 16 to 350 kDa, in particular 16 to 300 kDa, in particular 16 to 110 kDa, in particular 20 to 400 kDa, in particular 20 to 390 kDa, in particular 20 to 350 kDa, in particular 20 to 300 kDa, in particular 20 to 110 kDa, in particular 40 to 110 kDa, in particular 40 to 100 kDa, in particular 21 to 105 kDa, in particular 25 to 100 kDa, in particular 20 to 99 kDa, in particular 25 to 95 kDa, in particular 30 to 95 kDa, in particular 35 to 95 kDa.

Preferably, the recombinant type I collagen, in particular collagen peptide, has a molecular weight in a range of 16 to 25 kDa, in particular 16 kDa.

Preferably, the recombinant type I collagen, in particular collagen peptide, has a molecular weight in a range of 20 to 50 kDa, in particular 20 kDa.

Preferably, the recombinant type I collagen, in particular collagen peptide, has a molecular weight in a range of 40 to 50 kDa, in particular 45 kDa.

Preferably, the recombinant type I collagen, in particular collagen peptide, has a molecular weight in a range of 80 to 100 kDa, in particular 92 kDa.

In a particularly preferred embodiment, the natural type I collagen, in particular type I collagen peptide, can be a collagen peptide of type I with a molecular weight in a range of 16 to 400 Da, in particular 16 to 390 kDa, in particular 16 to 350 kDa, in particular 16 to 300 kDa, in particular 16 to 110 kDa, in particular 20 to 400 kDa, in particular 20 to 390 kDa, in particular 20 to 350 kDa, in particular 20 to 300 kDa, in particular 20 to 110 kDa, in particular 40 to 110 kDa, in particular 40 to 100 kDa, in particular 21 to 105 kDa, in particular 25 to 100 kDa, in particular 20 to 99 kDa, in particular 25 to 95 kDa, in particular 30 to 95 kDa, in particular 35 to 95 kDa.

Preferably, the natural type I collagen, in particular collagen peptide, has a molecular weight in a range of 16 to 25 kDa, in particular 16 kDa.

Preferably, the natural type I collagen, in particular collagen peptide, has a molecular weight in a range of 20 to 50 kDa, in particular 20 kDa.

Preferably, the natural type I collagen, in particular collagen peptide, has a molecular weight in a range of 40 to 50 kDa, in particular 45 kDa.

Preferably, the natural type I collagen, in particular collagen peptide, has a molecular weight in a range of 80 to 100 kDa, in particular 92 kDa.

In a particularly preferred embodiment, the recombinant or natural type I collagen, in particular the recombinant or natural type I collagen peptide, of the present invention is fully or partially hydroxylated, fully or partially glycolysed or fully or partially hydroxylated and glycolysed.

In a particularly preferred embodiment, the recombinant or natural type I collagen, in particular the recombinant or natural type I collagen peptide, according to the present invention is a non-hydroxylated type I collagen, in particular type I collagen peptide.

In a further preferred embodiment, the recombinant or natural type I collagen, in particular the recombinant or natural type I collagen peptide, according to the present invention is a hydroxylated type I collagen, in particular type I collagen peptide.

Preferably, the recombinant or natural type I collagen, in particular the recombinant prepared or natural type I collagen peptide, has hydroxylated proline and/or hydroxylated lysine.

Preferably, the recombinant or natural type I collagen, in particular the recombinant or natural type I collagen peptide, is a non-hydroxylated, partially hydroxylated or fully hydroxylated type I collagen, in particular type I collagen peptide.

According to a preferred embodiment of the present invention, the recombinant or natural type I collagen, in particular the recombinant or natural type I collagen peptide, is glycosylated.

More preferably, the recombinant or natural type I collagen, in particular the recombinant or natural type I collagen peptide, is glycosylated on at least one hydroxylated lysine. Preferably, each hydroxylated lysine of the recombinant or natural type I collagen, in particular the recombinant or natural type I collagen peptide, is glycosylated.

In a preferred embodiment of the present invention, the recombinant type I collagen, in particular recombinant type I collagen peptide, has no amino acid modification, in particular no hydroxylation. Particularly preferably, the recombinant type I collagen, in particular type I collagen peptide, has no hydroxylated and/or glycosylated amino acids.

Preferably, the type I collagen according to the invention, in particular type I collagen peptide, has an amino acid sequence occurring in type I collagen from vertebrates, in particular from fish, amphibians, reptiles, birds and mammals, in particular in pig, sheep, cattle, rodent, kangaroo, horse or from invertebrates, in particular jellyfish, in particular an amino acid sequence occurring in type I collagen from cattle.

Particularly preferably, the type I collagen, in particular type I collagen peptide, comprises the amino acid sequence according to SEQ ID No. 2, 4, 6 or 8. More preferably, the type I collagen, in particular type I collagen peptide, consists of the amino acid sequence according to SEQ ID No. 2, 4, 6 or 8.

In a further preferred embodiment of the present invention, the type I collagen, in particular type I collagen peptide, has at least 80%, preferably at least 85%, preferably at least 90%, preferably at least 95%, preferably at least 96%, preferably at least 97%, preferably at least 98%, preferably at least 99%, sequence identity with the amino acid sequence according to SEQ ID No. 2, 4, 6 or 8.

Preferably, the amino acid sequence of the recombinant type I collagen, in particular type I collagen peptide, is the amino acid sequence of a naturally occurring type I collagen, in particular type I collagen peptide. Preferably, the amino acid sequence of the recombinant type I collagen, in particular type I collagen peptide, is the amino acid sequence of a naturally non-occurring type I collagen, in particular type I collagen peptide. Preferably, the amino acid sequence of the recombinant type I collagen, in particular type I collagen peptide, is the amino acid sequence of a genetically modified type I collagen, in particular type I collagen peptide.

Particularly preferably, the type I collagen, in particular type I collagen peptide, according to the invention has an amino acid sequence that is present in non-human collagen, in particular in non-human type I collagen peptides, preferably in the al chain of non-human type I collagen, in particular an amino acid sequence occurring in bovine, porcine, equine, ovine, piscine or avian collagen, in particular an amino acid sequence occurring in bovine collagen.

In a preferred embodiment of the present invention, the recombinant type I collagen, in particular the recombinant type I collagen peptide, is collagenase resistant, in particular resistant to digestion by human collagenases.

According to a further preferred embodiment of the present invention, the recombinant or natural type I collagen, in particular type I collagen peptide, with a molecular weight of at least 16 kDa is capable of inducing oral tolerance, in particular to endogenously present collagen, in particular endogenously present type I collagen, in particular endogenous type I collagen present in skin or mucous membrane.

According to a further preferred embodiment of the present invention, the recombinant or natural type I collagen, in particular type I collagen peptide, with a molecular weight of at least 16 kDa is capable of suppressing the synthesis of immunoglobulins.

According to a further preferred embodiment of the present invention, the recombinant or natural type I collagen, in particular type I collagen peptide, with a molecular weight of at least 16 kDa is capable of suppressing the synthesis of pro-inflammatory cytokines.

According to a further preferred embodiment of the present invention, the recombinant or natural type I collagen, in particular type I collagen peptide, with a molecular weight of at least 16 kDa is capable of stimulating the synthesis of anti-inflammatory cytokines.

Preferably, the natural or recombinant type I collagens provided according to the invention, in particular recombinant type I collagen peptides, show an inducing effect on the differentiation of peripheral blood monocytes into immunosuppressive M2 macrophages.

In a further preferred embodiment, the recombinant or natural type I collagens provided according to the invention, in particular recombinant type I collagen peptides, lead to a reduction in the synthesis of pro-inflammatory cytokines, in particular TNFα and IFNγ, and/or to an inducing of the synthesis of anti-inflammatory cytokines, in particular IL-10, IL-4 and TGF-β, in particular IL-10.

According to a preferred embodiment of the present invention, the recombinant or natural type I collagens provided according to the invention, in particular recombinant type I collagen peptides, lead to a stimulation/inducing of the differentiation of naive CD4+T progenitor cells into T suppressor cells. Particularly preferably, the stimulation/inducing of the differentiation of naive CD4+T progenitor cells into T suppressor cells results in an increased release of anti-inflammatory cytokines, preferably IL-10, IL-4 and/or TGF-β.

In a preferred embodiment of the present invention, the recombinant or natural type I collagens provided according to the invention, in particular recombinant type I collagen peptides, cause a reduced expression of pro-inflammatory cytokines, preferably of IL-1β, IFNγ, TNFα and/or IL-6, in particular by dermal fibroblasts.

According to a further preferred embodiment of the present invention, the recombinant or natural type I collagen, in particular type I collagen peptide, with a molecular weight of at least 16 kDa is capable of suppressing the synthesis of immunoglobulins, suppressing the synthesis of pro-inflammatory cytokines and stimulating the synthesis of anti-inflammatory cytokines.

According to a preferred embodiment of the present invention, the recombinant or natural type I collagen, in particular type I collagen peptide, for use in a therapeutic method for oral therapy of inflammatory skin and mucous membrane diseases of a human or animal patient has a molecular weight in a range of 16 to 400 kDa, in particular 16 to 390 kDa, in particular 16 to 350 kDa, in particular 16 to 300 kDa, in particular 16 to 110 kDa, in particular 20 to 400 kDa, in particular 20 to 390 kDa, in particular 20 to 350 kDa, in particular 20 to 300 kDa, in particular 20 to 110 kDa, in particular 40 to 110 kDa, in particular 40 to 100 kDa, in particular 21 to 105 kDa, in particular 25 to 100 kDa, in particular 20 to 99 kDa, in particular 25 to 95 kDa, in particular 30 to 95 kDa, in particular 35 to 95 kDa.

According to a preferred embodiment of the present invention, the recombinant or natural type I collagen, in particular type I collagen peptide, for use in a therapeutic method for oral therapy of inflammatory skin and mucous membrane diseases of a human or animal patient has a molecular weight in a range of 35 to 95, in particular 40 to 92 kDa.

Preferably, the recombinant or natural type I collagen, in particular collagen peptide, has a molecular weight in a range of 16 to 25 kDa, in particular 16 kDa.

Preferably, the recombinant or natural type I collagen, in particular collagen peptide, has a molecular weight in a range of 20 to 50 kDa, in particular 20 kDa.

Preferably, the recombinant or natural type I collagen has a molecular weight in a range of 40 to 50 kDa, in particular 45 kDa.

Preferably, the recombinant or natural type I collagen has a molecular weight in a range of 80 to 100 kDa, in particular 92 kDa.

In a preferred embodiment of the present invention, the recombinant or natural type I collagen according to the invention, in particular type I collagen peptide, is used alone, i.e. in isolated form, i.e. without further substances, in particular without any further collagen, in the provided use according to the invention.

In a preferred embodiment of the present invention, the recombinant or natural type I collagen, in particular type I collagen peptide, according to the invention is present as a homogeneous preparation, in particular as a homogeneous preparation of a single recombinant or natural type I collagen, in particular type I collagen peptide, with a molecular weight of at least 16 kDa, in particular at least 20 kDa, in particular without any further collagen.

In a preferred embodiment of the present invention, the recombinant or natural type I collagen, in particular type I collagen peptide, according to the invention is present as a mixture of type I collagen, in particular type I collagen peptides, which all have a molecular weight of at least 16 kDa, in particular at least 20 kDa, in particular without any further collagen.

In a further embodiment of the present invention, the recombinant or natural type I collagen, in particular type I collagen peptide, according to the invention is used as the only biologically efficacy substance in the provided use according to the invention.

In a particularly preferred embodiment, the present invention relates to compositions comprising at least one type I collagen peptide, in particular at least one type I collagen peptide peptide, with a molecular weight of at least 16 kDa, in particular at least 20 kDa, which, in addition to the at least one recombinant or natural type I collagen, in particular the at least one recombinant or natural type I collagen peptide, and optionally a pharmaceutically acceptable and/or food safe carrier, do not contain any further substances, in particular without any further collagen.

In a particularly preferred embodiment, the composition having at least one type I collagen peptide, in particular at least one type I collagen peptide, with a molecular weight of at least 16 kDa, in particular at least 20 kDa, is present in a dosage form suitable for oral administration in a human or animal body.

The present invention also relates to a composition comprising at least one type I collagen peptide, in particular at least one type I collagen peptide, with a molecular weight of at least 16 kDa, in particular at least 20 kDa, according to the present invention and at least one pharmaceutically acceptable and/or food safe carrier and, optionally, at least one additive or excipient for use in a therapeutic method for oral therapy of inflammatory skin and mucous membrane diseases.

The present invention therefore also relates to a composition for use in a therapeutic method for the therapeutic prophylaxis or therapeutic treatment of immune intolerance reactions to type I collagen, in particular endogenous type I collagen, by inducing oral tolerance to type I collagen, in particular endogenous type I collagen.

The present invention also relates to a composition for use in a method for inducing oral tolerance to type I collagen, in particular endogenous type I collagen, wherein the composition leads to the inducing of oral tolerance in the human or animal body.

The present invention also relates to a composition comprising type I collagen peptide, in particular recombinant or natural type I collagen peptides, with a molecular weight of at least 16 kDa, in particular at least 20 kDa, in particular pharmaceutical compositions or dietary supplements, or food or luxury foods, for use for inducing oral tolerance to type I collagen, in particular endogenous type I collagen.

Compositions according to the invention for oral administration can in particular be pharmaceutical compositions, food supplements or food and luxury foods. In particular, the compositions according to the invention are pharmaceutical compositions. In particular, the compositions according to the invention are food supplements.

The present invention relates in particular to a pharmaceutical composition comprising a type I collagen according to the invention, in particular type I collagen peptide, with a molecular weight of at least 16 kDa, in particular at least 20 kDa, and at least one pharmaceutically acceptable carrier, and to the pharmaceutical composition for use in a therapeutic method for treating inflammatory skin and mucous membrane diseases of the human or animal body. Accordingly, it can be provided to administer the type I collagen according to the invention, in particular type I collagen peptide, in the form of a pharmaceutical composition. The pharmaceutical composition according to the invention is particularly advantageously administered, for example, in the form of tablets, lozenges, chewable tablets, powder, granules, hard capsules, soft capsules, capsules, bite capsules, dragees, pastilles, extrudates, juices, suspensions, gels or ointments.

In a particularly preferred embodiment of the present invention, the type I collagen used according to the invention, in particular type I collagen peptide, is present in a dosage form that enables sustained intestinal release, in particular a sustained-release capsule.

In a particularly preferred embodiment, the composition according to the invention is present in a form suitable for oral administration, in particular in a dose of 1 to 60 mg/day, in particular 5 to 50 mg/day, of type I collagen, in particular type I collagen peptide, with a molecular weight of at least 16 kDa, in particular at least 20 kDa.

The present invention further relates to a food supplement comprising a type I collagen according to the invention, in particular type I collagen peptide, with a molecular weight of at least 16 kDa, in particular at least 20 kDa, and at least one food safe carrier, and to the food supplement for use in a therapeutic method for the treatment of inflammatory skin and mucous membrane diseases of the human or animal body. Accordingly, it can be provided to administer the type I collagen according to the invention, in particular type I collagen peptide, in the form of a food supplement. Particularly advantageous, the food supplement according to the invention is present as a hard capsule, soft capsule, capsule, bite capsule, tablet, dragee, pastille, sachet, extrudate, solution, suspension or gel, for example in an ampoule, as granules or powder.

The invention also relates to a food or luxury food comprising a type I collagen according to the invention, in particular type I collagen peptide, with a molecular weight of at least 16 kDa, in particular at least 20 kDa, and to the food or luxury food for use in a therapeutic method for treating inflammatory skin and mucous membrane diseases of the human or animal body. According to a preferred embodiment, the food or luxury food is a chocolate bar, protein bar, cereal bar, instant powder for the preparation of beverages, milk, milk products, for example yoghurt, whey or curd and milk substitutes, for example soya milk, rice milk, almond milk and coconut milk, functional food or a beverage, for example refreshment or fitness drink.

If the recombinant type I collagen, in particular type I collagen peptide, according to a preferred embodiment of the invention is not to be used as the only biological efficacy component of a composition, in particular a pharmaceutical composition, a food supplement, or a food or luxury food, it can be combined with one or more further additives or excipients, in particular those which have a positive effect on general health, in particular on skin and mucous membrane health. Preferred excipients according to the invention are selected from the group consisting of vitamin C, vitamins of the B, D, E and K series, omega-3 fatty acids, omega-6 fatty acids, conjugated linolenic acids, caffeine and its derivatives, guarana extract, rose hip extract, green tea extract, polyphenols, epigallocatechin gallate, creatine, L-carnitine, α-lipoic acid, N-acetylcysteine, NADH, D-ribose, magnesium aspartate, antioxidants such as anthocyanins, carotenoids, flavonoids, resveratrol, glutathione and superoxide dismutase, minerals such as iron, magnesium, calcium, zinc, selenium and phosphorus, as well as other proteins, hydrolysates and peptides such as soya, wheat and whey protein.

In a particularly preferred embodiment, it can be provided that the composition according to the invention, in particular the pharmaceutical composition, the food supplement or the food or luxury food, has a corticosteroid, in particular a glucocorticoid, in particular cortisone.

In a further preferred embodiment, it can be provided that the composition according to the invention, in particular the pharmaceutical composition, the food supplement or a food or luxury food, has an additive, wherein the additive is a recombinantly produced collagen hydrolysate, a collagen hydrolysate derived from natural sources, a recombinantly produced type I collagen, a type I collagen recovered from a natural source or a combination thereof, in particular each with a molecular weight of at least 16 kDa, in particular at least 20 kDa.

In a further preferred embodiment of the present invention, the products according to the invention, in particular the pharmaceutical composition, the food supplement, or the food or luxury food, do not contain any further proteins or peptides, in particular no further collagen peptides, in addition to the type I collagen according to the invention, in particular type I collagen peptide.

The present invention also relates to methods for therapy, in particular for the prevention and/or treatment of inflammatory skin and mucous membrane diseases, according to which an amount sufficient for the therapeutic purpose of at least one of the recombinant or natural type I collagens according to the invention, in particular type I collagen peptides, with a molecular weight of at least 16 kDa, in particular at least 20 kDa, optionally with a carrier and, optionally, an excipient or additive, is orally administered to the human or animal body.

The present invention also relates to methods for inducing oral tolerance to type I collagen, in particular endogenous type I collagen, in a human or animal body, comprising the administration of an amount sufficient for a therapeutic purpose of at least one of the recombinant or natural type I collagens according to the invention, in particular recombinant or natural type I collagen peptides, with a molecular weight of at least 16 kDa, in particular at least 20 kDa, optionally by means of a carrier and, optionally, an excipient or additive, wherein the administration is carried out orally.

The present invention also relates to methods for the therapeutic treatment or therapeutic prophylaxis of immune intolerance to type I collagen, in particular type I collagen peptide, comprising the oral administration of an amount sufficient for a therapeutic purpose of at least one of the recombinant or natural type I collagens according to the invention, in particular recombinant or natural type I collagen peptides, with a molecular weight of at least 16 kDa, in particular at least 20 kDa, optionally by means of a carrier and, optionally, an excipient or additive.

The present invention also relates to the use of recombinant or natural type I collagen, in particular recombinant or natural type I collagen peptides, with a molecular weight of at least 16 kDa, in particular at least 20 kDa, for use in a non-therapeutic method for preservation of the skin and mucous membrane health of a human or animal, according to which a sufficient amount of at least one of the recombinant or natural type I collagens according to the invention, in particular type I collagen peptides, optionally with a carrier and, optionally, an auxiliary or additive, is orally administered to the human or animal body for the maintenance of skin and mucosal health. In this particularly preferred embodiment of the present invention, the human or animal has no skin or mucous membrane disease. Accordingly, in a particularly preferred embodiment, oral administration of recombinant or natural type I collagen, in particular recombinant type I collagen peptides, with a molecular weight of at least 16 kDa, in particular at least 20 kDa, to a human or an animal can be provided, which has no skin or mucous membrane disease and maintains its skin and mucous membrane health, in particular intestinal mucous membrane health, by administering the natural or recombinant type I collagen, in particular recombinant or natural type I collagen peptide.

In addition, the present invention relates to a method for preparing a recombinant type I collagen, in particular type I collagen peptide, useable according to the invention, with a molecular weight of at least 16 kDa, in particular at least 20 kDa, comprising the method steps:
a) providing an expression system which has at least one expression cassette, wherein the expression cassette has at least one nucleotide sequence which encodes a type I collagen, in particular type I collagen peptide, with a molecular weight of at least 16 kDa, in particular at least 20 kDa,
b) cultivating the expression system under conditions which enable the expression of the type I collagen, in particular type I collagen peptide, and
c) recovering the type I collagen according to the invention, in particular type I collagen peptide, with a molecular weight of at least 16 kDa, in particular at least 20 kDa.

The method provided according to the invention for the preparation of a recombinant type I collagen, in particular type I collagen peptide, useable according to the invention, with a molecular weight of at least 16 kDa, in particular at least 20 kDa, is characterised in particular by the fact that a precisely defined, recombinantly prepared type I collagen, in particular type I collagen peptide, is recovered, which, in particular due to its biological efficacy, is suitable for use in a therapeutic method for the treatment of inflammatory skin and mucous membrane diseases of the human or animal body or for maintaining skin or mucous membrane health.

The recombinant type I collagen provided according to the invention, in particular type I collagen peptide, with a molecular weight of at least 16 kDa, in particular at least 20 kDa, has a particularly high purity due to its recombinant production method compared to, for example, type I collagen, in particular type I collagen peptides, recovered hydrolytically from natural sources. It can also be provided on an industrial scale in a wide variety of expression systems without undesirable contamination, wherein the recombinant type I collagen, in particular type I collagen peptide, at the same time advantageously has biological efficacy.

Type I collagen peptide and its preparation are described, for example, in WO 2005/012356, WO 01/34646, WO 01/34647 and WO 01/34801. These documents disclose the obtaining of recombinant type I collagen peptides as well as hydroxylation and fibrillogenesis for obtaining procollagen in recombinant cell culture and are fully incorporated into the present disclosure with regard to the preparation of recombinant type I collagen as well as recombinant type I collagen peptides, in particular also in hydroxylated and triple-helical form.

The biological efficacy of the recombinant type I collagen found according to the invention, in particular of the type I collagen peptides, with a molecular weight of at least 16 kDa, in particular at least 20 kDa, and, connected with this, its or their suitability for use in a method for the therapeutic treatment of inflammatory skin and mucous membrane diseases of the human or animal body is advantageously already conferred in a preferred embodiment on the type I collagen, in particular type I collagen peptides, recovered directly from the method according to the invention, without the need for further processing steps. Thus, in a preferred embodiment, both the hydroxylated and the non-hydroxylated recombinant type I collagens, in particular type I collagen peptides, according to the present invention have a biological efficacy, in particular at least the same biological efficacy as type I collagen recovered from natural sources, and particularly preferably a better biological efficacy than type I collagen recovered from natural sources.

It is particularly advantageous here that the recombinant type I collagens according to the invention, in particular type I collagen peptides, surprisingly have a biological efficacy even in non-hydroxylated form, preferably the same biological efficacy as type I collagen recovered from natural sources, particularly preferably a better biological efficacy than type I collagen recovered from natural sources.

Preferably, both the hydroxylated and the non-hydroxylated recombinant type I collagens, in particular type I collagen peptides, according to the present invention shown biological efficacy, preferably at least the same biological efficacy as type I collagen recovered from natural sources, more preferably better biological efficacy than type I collagen recovered from natural sources.

Preferably, the expression system provided in step a) is a host cell, in particular a prokaryotic or eukaryotic cell.

Preferably, the expression system is a host cell selected from the group consisting of bacterial cell, yeast cell, fungal cell, mammalian cell, insect cell and plant cell.

Preferably, the expression system, in particular the host cell, is a bacterial cell, in particular of the species Escherichia coli or Bacillus subtilis.

In a further preferred embodiment, the expression system, in particular the host cell, is a yeast cell, in particular of the species Saccharomyces cerevisiae, Komagataella phaffi or Ogataea angusta (Hansenula polymorpha), in particular Komagataella phaffi.

Preferably, the expression system, in particular the host cell, is a fungal cell, in particular of the species Aspergillus niger.

In a further preferred embodiment of the present invention, the expression system, in particular the host cell, is a mammalian cell, in particular a CHO cell, a HeLa cell or a HEK293 cell.

Preferably, the expression system, in particular the host cell, is an insect cell, in particular an Sf-9, Sf-21 or Tn-5 cell.

Preferably, the expression system, in particular the host cell, is a plant cell, in particular a maize or tobacco cell.

In a further preferred embodiment of the present invention, the expression system provided in step a) is a host cell capable of hydroxylating proline, lysine or proline and lysine residues of the expressed collagen peptide. Preferably, the expression system provided in step a) is a host cell capable of hydroxylating proline, lysine or proline and lysine residues of the expressed collagen peptide.

Preferably, the expression system provided in step a) is an expression system having prolyl hydroxylase and/or lysyl hydroxylase activity. Preferably, the expression system provided in step a) is a host cell having prolyl hydroxylase and/or lysyl hydroxylase activity.

In a preferred embodiment, the expression system provided in step a) is a host cell having at least one expression cassette comprising a prolyl 4-hydroxylase-encoding polynucleotide sequence. Particularly preferably, the expression system provided in step a) is a host cell which has at least one expression cassette comprising a prolyl-4-hydroxylase-encoding polynucleotide sequence, so that in method step c) an in vivo hydroxylated type I collagen, in particular type I collagen peptide, is recovered.

In a preferred embodiment, the expression system provided in step a) is a host cell having at least one expression cassette comprising a lysylhydroxylase-encoding polynucleotide sequence.

Particularly preferably, the expression system provided in step a) is a host cell which has at least one expression cassette comprising a lysylhydroxylase-encoding polynucleotide sequence, so that in method step c) an in vivo hydroxylated type I collagen, in particular type I collagen peptide, is recovered.

In a further preferred embodiment of the present invention, the expression system provided in step a) is a host cell having at least one expression cassette comprising a prolyl-4-hydroxylase-encoding polynucleotide sequence and at least one expression cassette comprising a lysylhydroxylase-encoding polynucleotide sequence. Particularly preferably, the expression system provided in step a) is a host cell having at least one expression cassette comprising a prolyl-4-hydroxylase-encoding polynucleotide sequence and at least one expression cassette comprising a lysylhydroxylase-encoding polynucleotide sequence, so that an in vivo hydroxylated type I collagen, in particular type I collagen peptide, is recovered in method step c).

The present invention thus also relates to a method for preparing a recombinant type I collagen, in particular type I collagen peptide, useable according to the invention, in particular an in vivo hydroxylated type I collagen, in particular type I collagen peptide, with a molecular weight of at least 16 kDa, in particular at least 20 kDa, comprising the method steps
a) providing an expression system which has at least one expression cassette, wherein the expression cassette has at least one nucleotide sequence which encodes a type I collagen, in particular type I collagen peptide, with a molecular weight of at least 16 kDa, in particular at least 20 kDa, and wherein the expression system is capable of hydroxylating proline, lysine or proline and lysine residues of the expressed collagen peptide,
b) cultivating the expression system under conditions which enable the expression and hydroxylation of the type I collagen, in particular type I collagen peptide,
c) recovering the type I collagen, in particular type I collagen peptide, according to the invention, in particular the in vivo hydroxylated type I collagen, in particular type I collagen peptide with a molecular weight of at least 16 kDa, in particular at least 20 kDa.

With the aid of the aforementioned method, it is thus advantageously possible to obtain an in vivo hydroxylated recombinantly prepared type I collagen, in particular type I collagen peptide, with a molecular weight of at least 16 kDa, in particular at least 20 kDa, which is characterised by a specific pattern of post-translational modifications, in particular hydroxylations and glycosylations, depending on the cell-based expression system used. In this way, it is advantageously possible in particular to obtain directly, i.e. without the need for subsequent modification, a type I collagen, in particular a recombinantly prepared type I collagen peptide, with desired biological efficacy for use in a method for therapeutic treatment of inflammatory skin and mucous membrane diseases of the human or animal body.

In a preferred embodiment, the recombinant in vivo hydroxylated collagen peptide prepared according to the invention has a biological efficacy. According to a further embodiment of the present invention, the expression system provided in step a) is an expression system which is not capable of causing hydroxylation of proline, lysine or proline and lysine residues of the expressed collagen peptide, in particular the expression system provided in step a) does not have prolyl hydroxylase and lysyl hydroxylase activity.

Thus, the present invention comprises a method for preparing a recombinant collagen peptide useable according to the invention, in particular a non-hydroxylated collagen peptide, with a molecular weight of at least 16 kDa, in particular at least 20 kDa, comprising the method steps
a) providing an expression system which has at least one expression cassette, wherein the expression cassette has at least one nucleotide sequence which encodes a type I collagen, in particular type I collagen peptide, with a molecular weight of at least 16 kDa, in particular at least 20 kDa, and wherein the expression system is not capable of hydroxylating proline, lysine or proline and lysine residues of the expressed type I collagen, in particular type I collagen peptide,
b) cultivating the expression system under conditions which enable the expression of the type I collagen, in particular type I collagen peptide,
c) recovering the type I collagen, in particular type I collagen peptide, according to the invention, in particular the non-hydroxylated type I collagen, in particular type I collagen peptide with a molecular weight of at least 16 kDa, in particular at least 20 kDa.

According to a preferred embodiment of the present invention, the at least one nucleotide sequence of the at least one expression cassette is codon-optimised, which means that those codons in the nucleotide sequence which are not or not preferably used by the translation system of the provided expression system, in particular the provided cell-based expression system, in particular the provided host cell, are replaced by those which are preferably used by the translation system of the provided expression system, in particular the provided cell-based expression system, in particular the provided host cell, without thereby changing the amino acid sequence of the encoded peptide or protein.

In a preferred embodiment of the present invention, the type I collagen, in particular type I collagen peptide, encoded by the nucleotide sequence is a type I collagen, in particular type I collagen peptide, of a vertebrate, in particular a mammal, for example a human or a non-human mammal, for example a horse, kangaroo, rodent, pig, sheep or cattle, a bird, for example a chicken, a fish, an amphibian, a reptile or an invertebrate, for example a jellyfish.

In a preferred embodiment of the present invention, the expression cassette provided in step a) comprises at least one nucleotide sequence according to SEQ ID No. 1, 3, 5 or 7.

Particularly preferably, the expression cassette provided in step a) comprises at least one nucleotide sequence having a sequence identity of at least 90%, preferably at least 95%, preferably at least 96%, preferably at least 97%, preferably at least 98%, preferably at least 99%, to the nucleotide sequence according to SEQ ID No. 1, 3, 5 or 7.

Particularly preferably, the type I collagen, in particular type I collagen peptide, encoded by the nucleotide sequence is a type I collagen, in particular type I collagen peptide, comprising the amino acid sequence according to SEQ ID No. 2, 4, 6 or 8. Preferably, the type I collagen, in particular type I collagen peptide, encoded by the nucleotide sequence comprises the amino acid sequence according to SEQ ID No. 2, 4, 6 or 8.

In a further preferred embodiment of the present invention, the type I collagen, in particular type I collagen peptide, encoded by the nucleotide sequence has at least 80%, preferably at least 85%, preferably at least 90%, preferably at least 95%, preferably at least 96%, preferably at least 97%, preferably at least 98%, preferably at least 99%, sequence identity with the amino acid sequence according to SEQ ID No. 2, 4, 6 or 8.

Preferably, the type I collagen, in particular type I collagen peptide, encoded by the nucleotide sequence is a naturally occurring type I collagen, in particular type I collagen peptide. In a further preferred embodiment of the present invention, the type I collagen, in particular type I collagen peptide, encoded by the nucleotide sequence is not a naturally occurring type I collagen, in particular type I collagen peptide. Preferably, the type I collagen, in particular type I collagen peptide, encoded by the nucleotide sequence is a genetically engineered collagen peptide.

According to a preferred embodiment of the present invention, the at least one nucleotide sequence encodes a type I collagen peptide with a molecular weight in a range of 16 to 400 kDa, in particular 16 to 390 kDa, in particular 16 to 350 kDa, in particular 16 to 300 kDa, in particular 16 to 110 kDa, in particular 20 to 400 kDa, in particular 20 to 390 kDa, in particular 20 to 350 kDa, in particular 20 to 300 kDa, in particular 20 to 110 kDa, in particular 40 to 110 kDa, in particular 40 to 100 kDa, in particular 21 to 105 kDa, in particular 80 to 100 kDa, in particular 25 to 100 kDa, in particular 20 to 99 kDa, in particular 20 kDa, in particular 25 to 95 kDa, in particular 30 to 95 kDa, in particular 35 to 95 kDa, in particular 92 kDa, in particular from 40 to 50 kDa, in particular 45 kDa.

In a particularly preferred embodiment of the present invention, the methods according to the invention are characterised in that, in method step b), conditions are selected which enable the formation of a non-denatured, i.e. native, type I collagen, in particular type I collagen peptide.

In a particularly preferred embodiment of the present invention, the methods according to the invention are characterised in that, in method step b), conditions are selected which enable the formation of a triple-helical form of the type I collagen, in particular type I collagen peptide.

In a particularly preferred embodiment, the methods according to the invention can lead to the preparation of homogeneous and isolated preparations of specific type I collagen peptides with a molecular weight.

In a particularly preferred embodiment, the invention provides for also providing mixtures of such prepared isolated and homogeneous preparations of type I collagen peptides with a uniform molecular weight each.

The invention also provides for providing recombinant collagen peptide hydrolysates by lysis, in particular hydrolysis, from homogeneous and isolated type I collagen peptides with a uniform molecular weight prepared by means of the methods according to the invention. In particular, the present invention provides for providing both the homogeneously isolated type I collagen peptides with a uniform molecular weight, mixtures thereof or hydrolysates thereof for the oral therapy of inflammatory skin and mucous membrane diseases of a human or animal body according to the invention.

According to the present invention, the methods according to the invention are characterised in that, following method step b) or c), in a method step d), a type I collagen peptide hydrolysate is obtained by lysis, in particular hydrolysis, of the expressed type I collagen, in particular type I collagen peptide.

The type I collagen peptide hydrolysate obtained according to the invention by method step d) can be used either in the form of this type I collagen peptide hydrolysate or after isolation of one or more, preferably then homogeneously and isolated, type I collagen peptides as the type I collagen peptide according to the invention.

In a particularly preferred embodiment of the present invention, it can also be provided that homogeneous and isolated type I collagen peptides present are mixed with one another and thus represent a mixture of type I collagen peptides, wherein all type I collagen peptides have a molecular weight of at least 16 kDa, in particular at least 20 kDa.

In a particularly preferred embodiment, the invention therefore also relates to a type I collagen peptide which is present in isolated, homogeneous form having a uniform molecular weight, or a type I collagen peptide which is present in a mixture with recombinant or natural, in particular recombinant type I collagen peptides, or in a hydrolysate of a recombinant type I collagen, in particular recombinant type I collagen peptide.

The nucleotide sequences encoding the recombinant type I collagen peptide useable according to the invention can be obtained in a conventional manner, as described, for example, in WO 2005/012356, WO 01/34646, WO 01/34647 and WO 01/34801.

In a preferred embodiment of the present invention, the type I collagen, in particular type I collagen peptide, useable according to the invention, preferably prepared by one of the aforementioned methods according to the invention, with a molecular weight of at least 16 kDa, in particular at least 20 kDa, for use in a therapeutic method for the treatment of inflammatory skin and mucous membrane diseases of the human or animal body, is a non-hydroxylated, partially hydroxylated or fully hydroxylated type I collagen peptide, preferably a non-hydroxylated type I collagen peptide, preferably a partially hydroxylated type I collagen peptide, preferably a fully hydroxylated type I collagen peptide.

In a preferred embodiment of the present invention, the type I collagen, in particular natural or recombinant type I collagen peptide, useable according to the invention, preferably prepared by one of the above methods, with a molecular weight of at least 16 kDa, in particular at least 20 kDa, for use in a therapeutic method for treating inflammatory skin and mucous membrane diseases of the human or animal body is a glycosylated collagen peptide. Preferably, the type I collagen, in particular type I collagen peptide is glycosylated in vivo, more preferably glycosylated ex vivo.

In a further preferred embodiment of the present invention, the type I collagen, in particular type I collagen peptide, useable according to the invention, preferably prepared by one of the methods according to the invention, is a non-glycosylated type I collagen, in particular type I collagen peptide.

According to the invention, the term ‘biological efficacy’ is preferably understood to mean the ability of the type I collagens, in particular type I collagen peptides, useable according to the invention, to immunomodulate, in particular to regenerate immunosuppressive M2 macrophages, in particular from peripheral monocytes, and/or to regenerate immunosuppressive T suppressor cells, in particular from T precursor cells.

According to the invention, the term ‘biological efficacy’ is also preferably understood to mean the ability of the type I collagens, in particular type I collagen peptides, useable according to the invention, to suppress the formation and activity of pro-inflammatory cytokines, in particular TNFα, IL-6, IL-1β and IFNγ, or to stimulate the synthesis and activity of anti-inflammatory cytokines, in particular IL-4, IL-1β and TGF-β, in particular both.

According to the invention, the term ‘biological efficacy’ is preferably understood to mean that the type I collagens, in particular type I collagen peptides, useable according to the invention, are used for immunomodulation, in particular for the regeneration of immunosuppressive M2 macrophages, in particular from peripheral monocytes, and/or for the regeneration of immunosuppressive T-suppressor cells, in particular from T progenitor cells, for suppressing the formation of pro-inflammatory cytokines, in particular TNFα, IL-6, IL-1β and IFNγ, and for stimulating the synthesis of anti-inflammatory cytokines, in particular IL-4, IL-1β and TGF-β.

In a particularly preferred embodiment, the biological efficacy is determined in particular by means of detection methods known to the person skilled in the art for immunomodulating, in particular stimulating and suppressing, activities of substances and for anti-inflammatory and pro-inflammatory cytokines. In particular, the biological efficacy within the meaning of the present invention is determined by means of the method according to examples 2 to 4. According to the invention, the term ‘biological efficacy’ is preferably also understood to mean the ability of the type I collagens, in particular type I collagen peptides, useable according to the invention, to induce oral tolerance. In a particularly preferred embodiment, the presence of oral tolerance is determined by means of detection methods known to the person skilled in the art for determining the ability of a substance to induce oral tolerance, in particular by means of the method according to examples 2 to 4.

In the context of the present invention, pro-inflammatory cytokines are in particular TNFα, IL-6, IL-1β and IFN-gamma (IFNγ).

In the context of the present invention, anti-inflammatory cytokines are in particular IL-4, IL-10 and TGF-β.

In the context of the present invention, the term ‘suppression’ is understood to mean the partial or complete suppression of a synthesis of proteins, which can in particular take the form of a reduction or inhibition of protein synthesis or a reduction or inhibition of mRNA synthesis relating to the proteins.

In the context of the present invention, the term ‘collagen’ is understood in a manner customary in the art, in particular as defined, for example, in WO 01/34646. In a further preferred embodiment, the term ‘collagen’ is understood to mean a collagen protein or peptide having the sequence glycine-proline, glycine-4-hydroxyproline or glycine-X-4-hydroxyproline, preferably the repetitive motif (Gly-X-Y)n, wherein X and Y can be any amino acid, preferably are proline and 4-hydroxylproline. Particularly preferably, the term ‘collagen’ is understood to mean a peptide having the repetitive motif (Gly-Pro-Y)n and/or (Gly-X-Hyp)m, wherein X and Y can be any amino acid.

A ‘type I collagen’ according to the present invention is a collagen as in a manner customary in the art understood according to the foregoing, wherein the type I collagen has the amino acid sequence of a naturally occurring type I collagen, in particular the amino acid sequence of a type I collagen of a vertebrate, in particular pig, sheep, cattle, rodent, horse, bird, fish, reptile or amphibian or of an invertebrate, in particular jellyfish.

The type I collagen can be present as a monomeric collagen peptide, also referred to herein as a single-stranded collagen peptide, or as a di- or trimer, in particular a trimer, having at least two, in particular three collagen peptides, in particular of different or the same single-stranded collagen peptides. In particular, the type I collagen can be present as a triple-helical type I collagen peptide, in particular native type I collagen.

In the context of the present invention, the term ‘type I collagen peptide’ is understood to mean a single-stranded type I collagen peptide which has an amino acid sequence occurring in type I collagen as defined above, wherein the peptide is an oligopeptide or polypeptide. In particular, the type I collagen peptide can be present in chemically modified form, in particular hydroxylated and/or glycosylated form, or can be unmodified.

Preferably, the recombinant type I collagen used according to the invention, in particular type I collagen peptide, can have a sequence modification, in particular a function-preserving sequence modification of a naturally occurring type I collagen, in particular type I collagen peptide.

Accordingly, ‘type I collagen’ is also understood to mean a function-preserving sequence modification of a naturally occurring type I collagen, in particular type I collagen peptide, in particular if these have an amino acid sequence identity of at least 80% at amino acid level to the amino acid sequence of the naturally occurring type I collagen, in particular at least 85%, in particular at least 90%, in particular at least 95%, in particular at least 96%, in particular at least 97%, in particular at least 98%, in particular at least 99% amino acid sequence identity.

According to the invention, a type I collagen is present if the recombinant type I collagen either has exactly the amino acid sequence that occurs in a naturally occurring type I collagen or if a function-preserving sequence modification with an amino acid sequence identity of at least 80%, at least 85%, at least 90%, at least 91%, at least 92%, at least 93%, at least 94%, at least 95%, at least 96%, at least 97%, at least 98%, at least 99% compared to a naturally occurring type I collagen, in particular compared to naturally occurring type I collagen from a vertebrate, in particular pig, sheep, cattle, rodent, kangaroo, horse, a bird, a reptile, an amphibian or a fish or an invertebrate, in particular jellyfish, in particular if this amino acid identity is present compared to a naturally occurring type I collagen amino acid sequence from cattle.

In the context of the present invention, the amino acid sequence identity is determined using the Smith-Waterman algorithm (SSE2, Michael Farrar, 2006, 7.2 Nov. 2010) with parameters BL50 matrix (15:−5), Open/ext: −12/−2.

According to the invention, the term ‘function-preserving sequence modification’ is understood to mean the modification of a given, in particular naturally occurring amino acid sequence, in particular the replacement, addition and/or deletion of individual or several amino acids, which leads to an amino acid sequence deviating from the given amino acid sequence, but the modified amino acid sequence retains the function characteristic of the given amino acid sequence, in particular its biological efficacy.

Preferably, a ‘function-preserving sequence modification’ is understood to mean a modification of a given, in particular naturally occurring, amino acid sequence in which the function characteristic of the given amino acid sequence, in particular a biological efficacy, is retained at least 50%, preferably at least 60%, preferably at least 70%, preferably at least 80%, preferably at least 90%, preferably at least 95%, preferably 100%. Further preferably, according to the invention, a ‘function-preserving sequence modification’ is understood to mean a modification of a given amino acid sequence in which the modified amino acid sequence has at least 50%, preferably at least 55%, preferably at least 60%, preferably at least 65%, preferably at least 70%, preferably at least 75%, preferably at least 80%, preferably at least 85%, preferably at least 90%, sequence homology to the given amino acid sequence.

Particularly preferably, a sequence modification, in particular a ‘function-preserving sequence modification’ in the context of the present invention is a modification of a given, in particular naturally occurring, amino acid sequence, in which one or more amino acids having certain chemical-physical properties have been replaced by one or more amino acids each having the same or similar chemical-physical properties, in particular, for example, an amino acid having a non-polar side chain (for example, Ala, Val, Met, Leu, Ile, Pro, Trp, Phe) has been replaced by another amino acid having a non-polar side chain (for example, Ala, Val, Met, Leu, Ile, Pro, Trp, Phe), an amino acid with a polar, neutral side chain (for example Tyr, Thr, Gln, Gly, Ser, Cys, Asn) by another amino acid with a polar, neutral side chain (for example Tyr, Thr, Gln, Gly, Ser, Cys, Asn), an amino acid with an acidic side chain (for example Glu, Asp) is replaced by another amino acid with an acidic side chain (e.g. Glu, Asp) and/or an amino acid with a basic side chain (e.g. Lys, Arg, His) is replaced by another amino acid with a basic side chain (e.g. Lys, Arg, His). According to this embodiment, the chemical-physical properties of a given amino acid sequence are retained in the ‘function-preserving sequence modification’ or change only slightly.

In a further embodiment, it can be provided that the sequence modification, in particular a ‘function-preserving sequence modification’, which consists in the fact that at least one amino acid of a given amino acid sequence, in particular a naturally occurring amino acid sequence, preferably at least one non-essential amino acid, in particular Ala, Asn, Asp, Glu, Ser, of the given amino acid sequence, in particular the naturally occurring amino acid sequence, has been replaced by at least one very specific amino acid, in particular at least one essential amino acid, in particular Ile, Leu, Lys, Met, Phe, Thr, Trp, Val, His, Cys, Tyr, particularly preferably Trp, wherein the function characteristic of the given amino acid sequence, in particular the naturally occurring amino acid sequence, in particular the biological efficacy, in particular the biological efficacy according to the present invention, in particular the evidence shown in Example 2, is retained.

According to the invention, a sequence modification, in particular a ‘function-preserving sequence modification’, is also understood to mean the modification of a given amino acid sequence, in particular a naturally occurring amino acid sequence, which consists in the fact that at least one amino acid, preferably at least one essential amino acid, in particular Ile, Leu, Lys, Met, Phe, Thr, Trp, Val, His, Cys, Tyr, particularly preferably Trp has been added to the given amino acid sequence, in particular the naturally occurring amino acid sequence, wherein the function characteristic of the given amino acid sequence, in particular the naturally occurring amino acid sequence, is retained, in particular the biological efficacy of the present invention, in particular the biological efficacy according to the proof shown in Example 2. It can be provided according to the invention to add the at least one amino acid, preferably the at least one essential amino acid, in particular Ile, Leu, Lys, Met, Phe, Thr, Trp, Val, His, Cys, Tyr, particularly preferably Trp, N-terminally, C-terminally and/or within the amino acid sequence.

In the context of the present invention, the term ‘amino acid modification’ means a chemical modification of one or more amino acids, which can have taken place before, after or during the synthesis of the recombinant type I collagen, in particular type I collagen peptide, while retaining the original amino acid backbone, in particular one or more proteinogenic amino acids, of the type I collagen peptide. Thus, the term comprises both the use of chemically modified amino acids to synthesise the type I collagen according to the invention, in particular type I collagen peptide, and the chemical modification of the amino acids after or during the synthesis of the type I collagen, in particular type I collagen peptide. Typical amino acid modifications for collagen peptides are in particular hydroxylations at a proline and lysine residue as well as glycosylations of hydroxylated lysine residues. However, according to the invention, the term also comprises other chemical modifications of amino acids, such as phosphorylations, N-glycosylations, acetylations, methylations or myristoylations.

In the context of the present invention, a recombinant type I collagen, in particular type I collagen peptide, or a recombinantly prepared type I collagen, in particular type I collagen peptide, is understood to mean a type I collagen, in particular type I collagen peptide, recovered by biotechnological recombinant preparation using an expression system. According to the invention, the recombinant type I collagen, in particular type I collagen peptide, or the recombinantly prepared type I collagen, in particular type I collagen peptide, have in common that they are not recovered from natural sources.

In a particularly preferred embodiment of the present invention, the recombinant type I collagen, in particular type I collagen peptide, is present in the form of a homogeneous preparation of this type I collagen or type I collagen peptide, in particular such a preparation comprises at least 90 wt.-%, preferably at least 95 wt.-%, in particular at least 98 wt.-%, in particular at least 99 wt.-%, preferably 100 wt.-% of the type I collagen or type I collagen peptide. In a preferred embodiment, only type I collagens or type I collagen peptides of a certain specific size, i.e. of a certain molecular weight, i.e. of a single molecular species, in particular with the identical amino acid sequence, are present in a homogeneous preparation. In a preferred embodiment of the present invention, the recombinant type I collagen or the type I collagen peptide is present in isolated form. In a particularly preferred embodiment of the present invention, the recombinant type I collagen or type I collagen peptide is free from other proteins or peptides, in particular free from other substances, for example from impurities, in particular free from non-protein material, free from salts and/or free from other proteins or peptides.

In a particularly preferred embodiment of the present invention, the recombinant type I collagen or the type I collagen peptide with a molecular weight of at least 16 kDa is free of collagen, in particular also free of collagen peptides with a molecular weight of less than 16 kDa.

In a particularly preferred embodiment of the present invention, the natural type I collagen, in particular type I collagen peptide, is present in the form of a homogeneous preparation of this type I collagen or type I collagen peptide, in particular such a preparation comprises at least 90 wt.-%, preferably at least 95 wt.-%, in particular at least 98 wt.-%, in particular at least 99 wt.-%, preferably 100 wt.-% of the type I collagen or type I collagen peptide. In a preferred embodiment, only type I collagens or type I collagen peptides of a certain specific size, i.e. of a certain molecular weight, i.e. of a single molecular species, in particular with the identical amino acid sequence, are present in a homogeneous preparation. In a preferred embodiment of the present invention, the natural type I collagen or the type I collagen peptide is present in isolated form. In a particularly preferred embodiment of the present invention, the natural type I collagen or type I collagen peptide is free from other proteins or peptides, in particular free from other substances, for example from impurities, in particular free from non-protein material, free from salts and/or free from other proteins or peptides.

In a particularly preferred embodiment of the present invention, the natural type I collagen or type I collagen peptide, with a molecular weight of at least 16 kDa, is free from collagen, in particular also free from collagen peptides, with a molecular weight of less than 16 kDa.

In the context of the present invention, the molecular weight is preferably determined by gel permeation chromatography.

In the context of the present invention, the term ‘gelatin’ is understood in a manner customary in the art, in particular as defined, for example, in WO 01/34646.

In the context of the present invention, the term ‘recombinant DNA’ refers to an artificially prepared or manipulated DNA molecule prepared in vitro by genetic engineering methods. In a preferred embodiment, the recombinant DNA is composed of components of different organisms of origin.

In the context of the present invention, the term ‘expression cassette’ is understood to mean a DNA segment which is responsible for the transcription of the information encoded in this segment into an RNA, in particular into an mRNA, and which has at least one promoter and a protein-coding nucleotide sequence, generally at least one promoter, at least one protein-coding nucleotide sequence and optionally a terminator.

In the context of the present invention, a ‘nucleotide sequence’ is understood to mean the sequence, in particular continuous sequence, of the nucleotides of a nucleic acid, in particular a nucleic acid strand, in particular a DNA or RNA strand. A ‘nucleotide sequence’ is therefore to be understood both as an informational unit and as the DNA or RNA strand physically manifesting this information.

In the context of the present invention, an ‘expression system’ is understood to be a system in which targeted and controlled protein biosynthesis can take place. According to the invention, the term ‘expression system’ comprises both cell-free expression systems, in which the components necessary for protein biosynthesis are not present within a cell, i.e. protein biosynthesis takes place outside a cell, and cell-based expression systems, in which protein biosynthesis takes place within a living cell. In the context of the present invention, a cell-free expression system is preferably a lysate or an extract from E. coli, insect cells, wheat germ, tobacco cells or mammalian cells, in particular CHO cells or reticulocytes from rabbits, which has the components necessary for protein biosynthesis, in particular a translation and a transcription system. In the case of the use of a cell-free expression system in one of the methods according to the present invention, the term ‘cultivating’ is synonymous with ‘incubating’.

In the context of the present invention, a ‘host cell’ is understood to be a living cell capable of expressing peptides or proteins encoded in foreign DNA, in particular recombinant DNA.

According to the invention, the term ‘recovering the type I collagen, in particular type I collagen peptide’, according to method step c) is understood to mean a method known to the person skilled in the art for isolating the type I collagen or type I collagen peptide from a composition containing several components by means of known isolation methods, such as, for example, centrifugation methods, in particular differential centrifugation and/or density gradient centrifugation, chromatographic methods, in particular gel filtration, ion exchange, affinity and/or high-performance liquid chromatography, electrophoresis methods, filtration methods and/or extraction methods, wherein enrichment and purification of the component in question from the composition containing several components can preferably be achieved by sequential application of several isolation methods. If necessary, a cleavage of C- and/or N-terminal procollagen fragments can be carried out before, after or during the extraction to obtain collagen.

Fibrillogenesis, chemical modifications and secretion of the expressed type I collagen peptide can preferably also take place within the scope of the conditions of method step b).

According to the invention, ‘conditions which enable the expression of type I collagen, in particular type I collagen peptide’ are understood to mean conditions, such as in particular temperature, pressure, time, light and the presence or absence of inducers and/or repressors, which activate or enhance the expression of type I collagen, in particular type I collagen peptide. In a preferred embodiment, the expression of the type I collagen, in particular type I collagen peptide, takes place within the scope of a high cell density fermentation, in particular under high pressure, preferably high air pressure. The specific conditions that enable expression of the type I collagen, in particular type I collagen peptide, are known to the person skilled in the art and depend on the expression system used and the expression cassette used, in particular the promoter contained therein. The expression of type I collagen, in particular type I collagen peptide, can be constitutive or inducible expression, depending on the structure of the expression cassette.

In the context of the present invention, a therapeutic method for the oral therapy of ‘inflammatory skin and mucous membrane diseases’ is understood to mean a method for the prevention and/or treatment of inflammatory skin and mucous membrane diseases, in particular for the treatment of immunomodulated inflammatory skin and mucous membrane diseases, in particular intestinal mucous membrane diseases, wherein the administration of the type I collagen is carried out orally.

Inflammatory skin and mucous membrane diseases within the meaning of the present invention are, in particular, skin and mucous membrane diseases caused by autoimmune reactions, in particular by excessive immune reactions, in particular of the skin, such as cutaneous lupus erythematosus, dermatomyositis, lichen sclerosus, neurodermatitis, psoriasis, rosacea, lichen ruber, bullous pemphigoid, pemphigus vulgaris and acne as well as of the mucous membrane, in particular the intestinal mucous membrane, such as Crohn's disease, coeliac disease or ulcerative colitis.

According to the invention, a therapeutic method for the oral therapy of ‘inflammatory skin and mucous membrane diseases’ is preferably also understood to mean a method for inducing oral tolerance to endogenous collagen, in particular endogenous type I collagen, in particular endogenous type I collagen present in or on skin or mucous membrane tissue.

In the context of the present invention, ‘immunomodulated’ inflammatory skin and mucous membrane diseases is understood to mean that the inflammatory skin and mucous membrane diseases are caused in whole or at least in part by autoimmune diseases and/or immune intolerance, in particular to type I collagen.

In the context of the present invention, the terms ‘comprising’ and ‘having’ are understood to mean that, in addition to the elements explicitly comprised by these terms, further elements not explicitly mentioned can be added. In the context of the present invention, these terms are also understood to mean that only the explicitly mentioned elements are included and that no further elements are present. In this particular embodiment, the meaning of the terms ‘comprising’ and ‘having’ is synonymous with the term ‘consisting of’. Furthermore, the terms ‘comprising’ and ‘having’ also encompass compositions which, in addition to the explicitly mentioned elements, also contain further elements which are not mentioned but which are of a functionally and qualitatively subordinate nature. In this embodiment, the terms ‘comprising’ and ‘having’ are synonymous with the term ‘consisting essentially of’.

Where, in the context of the present invention, the first and second decimal places or the second decimal place are/is not indicated, these are/is to be set as 0.

In the context of the present invention, the term ‘and/or’ is understood to mean that all members of a group which are connected by the term ‘and/or’ are disclosed in any combination, both alternatively to each other and cumulatively to each other. For the expression ‘A, B and/or C’, this means that the following disclosure content is to be understood: a) A or B or C or b) (A and B) or c) (A and C) or d) (B and C) or e) (A and B and C).

Further preferred embodiments are shown in the sub-claims.

The invention is described below, without limiting the general idea of the invention, with reference to exemplary sequences, embodiments using the same and the associated figures.

Hereby designated:
SEQ ID No. 1: The coding nucleotide sequence of a 16 kDa bovine type I recombinant collagen peptide (CP16) (type COL1A1).
SEQ ID No. 2: The amino acid sequence of the collagen peptide according to SEQ ID No. 1.
SEQ ID No. 3: The coding nucleotide sequence of a 20 kDa bovine type I recombinant collagen peptide (CP20) (type COL1A1).
SEQ ID No. 4: The amino acid sequence of the collagen peptide according to SEQ ID No. 3.
SEQ ID No. 5: The coding nucleotide sequence of a 45 kDa bovine type I recombinant collagen peptide (CP45) (type COL1A1).
SEQ ID No. 6: The amino acid sequence of the collagen peptide according to SEQ ID No. 5.
SEQ ID No. 7: The coding nucleotide sequence of a 92 kDa bovine type I recombinant collagen peptide (CP90) (type COL1A1).
SEQ ID No. 8: The amino acid sequence of the collagen peptide according to SEQ ID No. 7.
SEQ ID No. 9: The amino acid sequence of an 8 kDa (7912 Da) hydroxylated bovine control collagen peptide (hereinafter referred to as CP9).

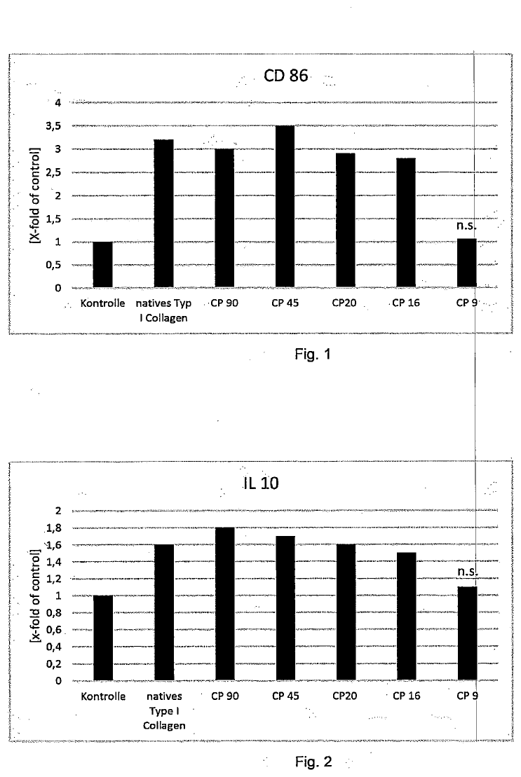

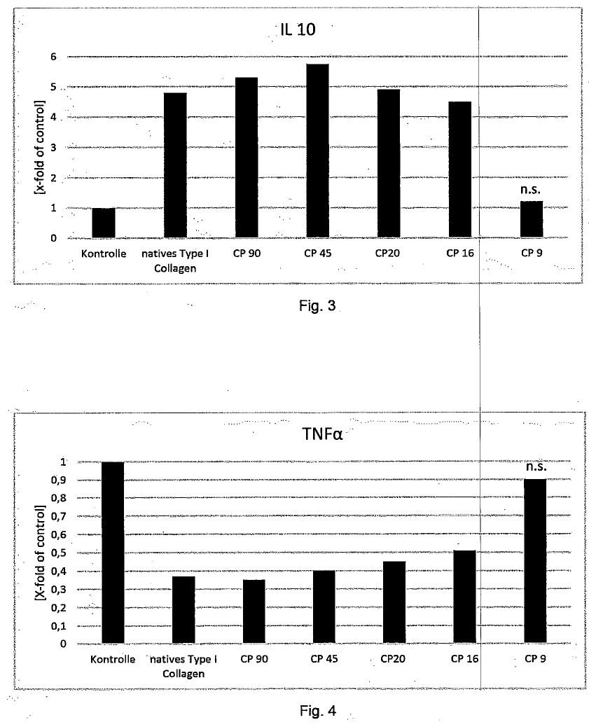

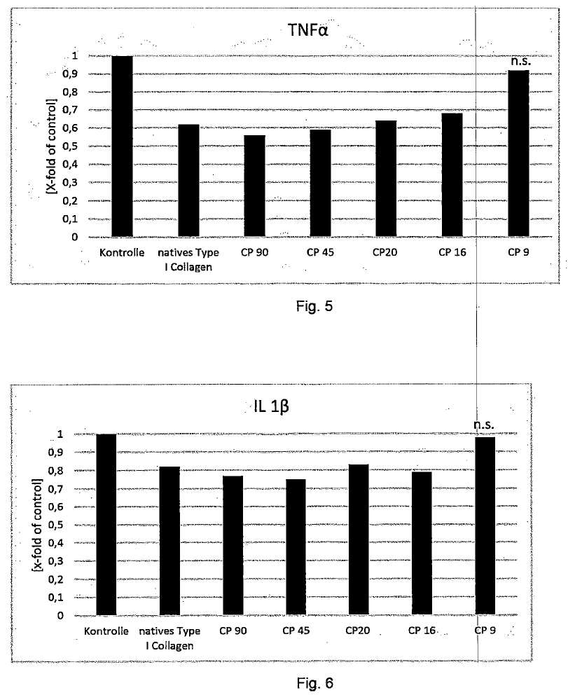

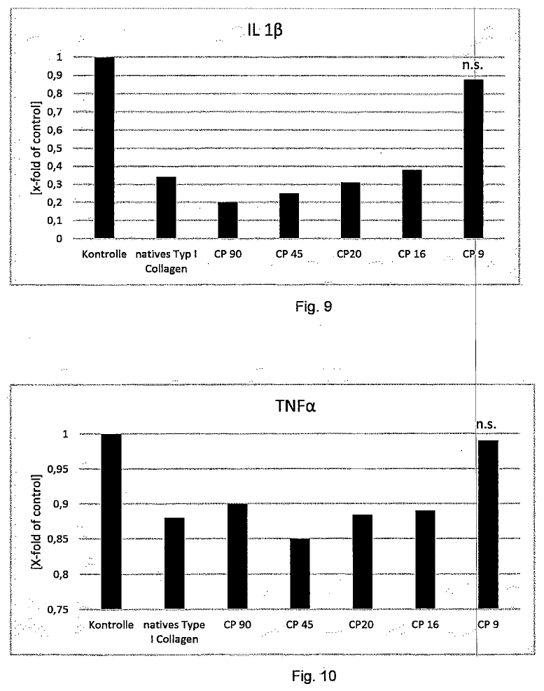

FIGS. 1 to 11 show in graphic form the stimulation or inhibition of the formation of anti- and pro-inflammatory cytokines and the surface marker CD86 in M2 macrophages (FIGS. 1 and 2), differentiated macrophages (FIGS. 3 and 4), T-suppressor cells (FIGS. 5 to 8) and dermal fibroblasts (FIGS. 9 to 11).

### EXAMPLE 1: PREPARATION OF RECOMBINANT TYPE I COLLAGEN

Recombinantly prepared hydroxylated bovine type I collagen peptides with the designations CP16, CP20, CP45 and CP90 and with the amino acid sequences according to SEQ ID No. 2, 4, 6 and 8 were recovered by recombinant expression of each of an expression cassette having the nucleotide sequence according to SEQ ID No. 1, 3, 5 or 7 in a Komagataella phaffi strain capable of hydroxylating proline residues. In the same way, an experimental control was provided in the form of the collagen peptide CP9 (SEQ ID No. 9) known from WO 2020/127929.

The strains used in each case for the recombinant expression of the collagen peptides were obtained by genomic integration of the coding nucleotide sequences of the respective bovine type I collagen peptides or the coding nucleotide sequence of a monomeric prolyl-4-hydroxylase from the mimivirus (P4H).

### EXAMPLE 2: PROOF OF CONCEPT—IMMUNE AND CYTOKINE MODULATION BY TYPE I COLLAGEN PEPTIDES ACCORDING TO EXAMPLE 1

The following studies were carried out on immune system cells downstream of Peyer's plaques.

The inner layer of the intestine consists of mucoid fluid-secreting enterocytes and an underlying lamina propria of loose connective tissue. The mucous membrane is infiltrated by gut-associated lymphoid tissue (GALT), which is part of the immune system and is involved in protecting the body from the invasion of pathogens into the gut.

The GALT consists of mesenteric lymph nodes of lymphoid tissue, the so-called Peyer's plaques. The lymphoid follicles are surrounded by an epithelial layer and specialised microfolded M cells, which are characterised by a reduced microvilli border and a glycocalyx and are exposed to the inside of the intestine.

M cells are specialised in the uptake of macromolecules such as soluble proteins, peptides, commensal and pathogenic microorganisms and viruses. The Peyer's plaques recognise and evaluate these macromolecules after transcytosis. Depending on the antigenic compound, they switch the body's immune response on or off. Pathogens are delivered to mononuclear phagocytes and lymphocytes located in the basolateral membrane of the M cells.

Native type I collagen is recognised by Peyer's patches via its ‘active’ epitopes and the additional glycopeptide side chains of the collagens.

Native type I collagen activates macrophages and dendritic cells, which then activate T cell precursors in the lymphoid follicles to differentiate into regulatory T cells of type I collagen.

Mature T cells then enter the bloodstream via the thoracic duct and, after reaching the target tissue, release mediators that attenuate the immune response in the tissue by releasing anti-inflammatory cytokines, IL-10, IL-4 and TGF-β. In addition, the pro-inflammatory cytokine TNF-α is downregulated in the target tissue.

Studies on the direct effect of collagen peptides on Peyer's patches are very difficult to realise experimentally and are fraught with a number of methodological problems. For this reason, the effect of the peptides to be tested was investigated on downstream cell systems of the immune system and thus the effect on specific immune reactions was demonstrated.

The immunomodulatory effect of the recombinantly prepared type I collagen peptides according to example 1 was determined in human peripheral blood monocytes (PBMC). Commercially obtained cells (3H10-25) from 3H-Biomedical AB, (Sweden) were used for this purpose.

The PBMC cells were first cultivated in macrophage-based medium DXF (C-28057, PromoCell, Germany) in cell culture flasks coated with human fibronectin (C-43060, PromoCell, Germany). The culture medium was supplemented with the associated supplement mix (supplement to C-28055, PromoCell, Germany) as well as 1% amphotericin and 1% penicillin-streptomycin. After a static cultivation period of 4-24 hours, dead, non-adherent monocytes were decanted. A polarisation of adherent monocytes to immunosuppressive macrophages of type 2b or 2c was induced by the addition of 4 μg/mL of recombinant type I collagen each. The cells were incubated for 6 days each with the samples to be analysed. The activated macrophages were then polarised by adding 1 μg/mL lipopolysaccharides from Escherichia coli (LPS, L6529, Merck, Germany).

After polarisation, the differentiation pattern of the generated macrophages was examined using specific cell differentiation markers (CDs). The differentiation of monocytes into inflammation-inducing M1 macrophages or immunosuppressive M2 macrophages was detected using specific markers. For this purpose, the M2 surface markers CD86, CD14 and CD163 were determined using ELISA (‘enzyme-linked immunosorbent assay’). The proof of each was performed for CD86 (850590096 Diaclone, Hölzel Diagnostics, Germany), CD14 (850780096 Diaclone, Hölzel Diagnostics, Germany) and CD163 (ELH-CD163 RayBiotech, Hölzel Diagnostics) exactly according to the manufacturer's instructions. To rule out differentiation to inflammatory M1 macrophages, the M1 macrophage markers CD86 (850590096 Diaclone, Hölzel Diagnostics, Germany) and CD80 (EK0707 Boster PicoKine, Hölzel Diagnostics, Germany) were also analysed.

In addition, the effect of type I collagen peptides on the formation of pro-inflammatory (TNFα, IFNγ) and anti-inflammatory cytokines (IL-10) in the culture supernatant was investigated. TNF (EK0525 Boster PicoKine, Hölzel Diagnostics, Germany), IL-10 (950060096, Diaclone, Hölzel Diagnostics, Germany) and IFNΥ (EK0373, Boster PicoKine, Hölzel Diagnostics, Germany) were determined using the ELISA technique according to the manufacturer's instructions.

The differentiation of monocytes into M1 or M2 macrophages was checked using a special differentiation medium (C-28055, PromoCell, Germany) and specific cultivation additives. The monocytes were, instead of described above, cultivated with macrophage differentiation medium (C-28055, PromoCell, Germany), which a GM-CSF supplementation mix (C60420A, PromoCell, Germany) to indicate M1 differentiation or a M-CSF GM-CSF supplementation mix (C60442A, PromoCell, Germany) to indicate M2 differentiation was added.

The type I collagen peptides CP90 (molecular weight approximately 92 kDa), CP45 (molecular weight approximately 45 kDa), CP20 (molecular weight approximately 20 kDa), CP16 (molecular weight approximately 16 kDa) and CP9 (molecular weight approximately 8 kDa) described above were used for the tests.

The data determined and presented below showed a statistically significant, advantageous effect of the tested 92, 45, 20 and 16 kDa collagen peptides on the regeneration of immunosuppressive M2 macrophages from peripheral blood monocytes.

The studies were carried out on M2 macrophages. In FIG. 1 and Table 1 the mean values (n=6) based on untreated control cells with regard to the surface marker CD86 are presented. Unless otherwise stated, each measured value was statistically significantly (p<0.05) different from the controls. n.s.=not significant based on the control group.

TABLE 1

| | Native type | | | | | |
|---|---|---|---|---|---|---|
| Control | I collagen | CP 90 | CP45 | CP 20 | CP 16 | CP 9 |
| 1 | 3.20 | 3.00 | 3.50 | 2.90 | 2.80 | 1.06 |

In FIG. 2 and table 2 the mean values (n=6) based on untreated control cells with regard to IL-1β are presented. Unless otherwise stated, each measured value was statistically significantly (p<0.05) different from the controls. n.s.=not significant based on the control group.

TABLE 2

| | Native type | | | | | |
|---|---|---|---|---|---|---|
| Control | I collagen | CP 90 | CP45 | CP 20 | CP 16 | CP 9 |
| 1 | 1.6 | 1.8 | 1.7 | 1.6 | 1.5 | 1.1 |

An up to 3.5-fold increase in the M2 macrophage surface marker CD86 (FIG. 1) and a 1.8-fold increase in the synthesis of the anti-inflammatory cytokine IL-1β (FIG. 2) showed the statistically significant, advantageous effect of the tested 92, 45, 20 and 16 kDa collagen peptides on the regeneration of immunosuppressive M2 macrophages from peripheral blood monocytes compared to the control. In contrast, in both studies the application of the shorter collagen peptide CP9 (8.0 kDa) showed no statistically significant effect compared to the untreated control group.

In differentiated macrophage cells, the cytokine synthesis profile showed an anti-inflammatory effect of the investigated type I collagen peptides. In addition, the synthesis of inflammatory cytokines was suppressed and the synthesis of anti-inflammatory IL-1β was induced.

The studies described in the following were carried out on differentiated macrophages. In FIG. 3 and Table 3 the mean values (n=6) based on untreated control cells with regard to IL-1β are presented. Unless otherwise stated, each measured value was statistically significantly (p<0.05) different from the controls. n.s.=not significant based on the control group.

TABLE 3

| | Native type | | | | | |
|---|---|---|---|---|---|---|
| Control | I collagen | CP 90 | CP45 | CP 20 | CP 16 | CP 9 |
| 1 | 4.80 | 5.30 | 5.75 | 4.90 | 4.50 | 1.20 |

In differentiated macrophages, a statistically significant anti-inflammatory effect of the investigated type I collagen peptides could be shown by an up to 5.75-fold increase in the synthesis of the IL-1β cytokine (FIG. 3). In contrast, the CP9 peptide showed only a very weak anti-inflammatory effect.

The proven anti-inflammatory effect of the peptides tested, as demonstrated by the IL-1β data presented above, was confirmed in a further experiment.

The studies described below were also carried out on differentiated macrophages. In FIG. 4 and table 4 the mean values (n=6) based on untreated control cells with regard to TNF-α are presented. Unless otherwise stated, each measured value was statistically significantly (p<0.05) different from the controls. n.s.=not significant based on the control group.

TABLE 4

| | Native type | | | | | |
|---|---|---|---|---|---|---|
| Control | I collagen | CP 90 | CP45 | CP 20 | CP 16 | CP 9 |
| 1 | 0.37 | 0.35 | 0.40 | 0.45 | 0.51 | 0.90 |

The application of the collagen peptides tested leaded to a statistically significant, up to 0.35-fold reduction of the inflammatory cytokine TNFα (FIG. 4) With the exception of CP9, all peptides tested had a clear TNFα-reducing effect.

### EXAMPLE 3: PROOF OF CONCEPT—STIMULATION OF NAIVE CD4⁺T PROGENITOR CELLS BY TYPE I COLLAGEN PEPTIDES ACCORDING TO EXAMPLE 1

After induction of monocytes into M2 macrophages by CP90, CP45, CP20 and CP16 (example 2), the macrophage base medium DXF (C-28057, PromoCell, Germany) was exchanged for T cell culture medium (3H800-50-50, 3H Biomedical AB, Sweden). Naive CD4⁺T progenitor cells (3H31-k, 3H Biomedical AB, Sweden) were added to the differentiated M2 macrophages. Through direct cell-cell contacts of naive T progenitor cells and the differentiated M2 macrophages and their cytokine cocktail, the T progenitor cells differentiate into collagen-specific (CP90, CP45, CP20 or CP16) regulatory T suppressor cells.

The mature T-suppressor cells were enriched with the ARTE (antigen-reactive T-cell enrichment) method, as specific T-cell clones are only formed at a low frequency. The specification of the T cells is determined by labelling the cells with cell surface marker (CD) antibodies coupled to various dyes such as biotin or phycoerythrin, followed by anti-biotin and anti-PE MicroBeads. The magnetic MicroBeads serve the separation and collection of specific T cell clone types. Afterwards, the T cells can be stained with fluorochrome-conjugated antibodies and quantified by flow cytometry. T suppressor cells are identified using forkhead box p3 (FoxP3) and CD25.

After differentiation, T suppressor cells secrete an anti-inflammatory cytokine cocktail of IL-10, IL-4 and TGF-β.

The data determined showed a statistically significant, advantageous effect of the tested collagen peptides CP90, CP45, CP20 and CP16 for the regeneration of immunosuppressive T-suppressor cells.

The differentiation of T-suppressor cells was demonstrated by the expression of forkhead box p3 (FoxP3). The effect of the CP90, CP45, CP20 and CP16 collagen peptides mediated via the M2 macrophages was demonstrated by the reduced production of pro-inflammatory cytokines by the T suppressor cells.

The studies shown in FIGS. 5 to 8 were carried out at T-suppressor cells. In FIG. 5 and table 5 the mean values (n=6) based on untreated control cells with regard to TNF-α are presented. Unless otherwise stated, each measured value was statistically significantly (p<0.05) different from the controls. n.s.=not significant based on the control group.

TABLE 5

| | Native type | | | | | |
|---|---|---|---|---|---|---|
| Control | I collagen | CP 90 | CP45 | CP 20 | CP 16 | CP 9 |
| 1 | 0.62 | 0.56 | 0.59 | 0.64 | 0.68 | 0.92 |

In FIG. 6 and table 6 the mean values (n=6) based on untreated control cells with regard to IL-1β are presented. Unless otherwise stated, each measured value was statistically significantly (p<0.05) different from the controls. n.s.=not significant based on the control group.

TABLE 6

| | Native type | | | | | |
|---|---|---|---|---|---|---|
| Control | I collagen | CP 90 | CP45 | CP 20 | CP 16 | CP 9 |
| 1 | 0.82 | 0.77 | 0.75 | 0.83 | 0.79 | 0.98 |

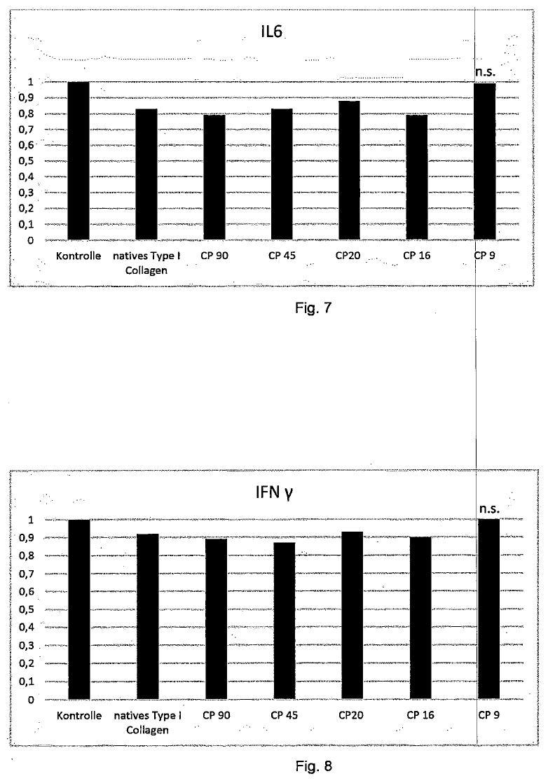

In FIG. 7 and table 7 the mean values (n=6) based on untreated control cells with regard to IL-6 are presented. Unless otherwise stated, each measured value was statistically significantly (p<0.05) different from the controls. n.s.=not significant based on the control group.

TABLE 7

| | Native type | | | | | |
|---|---|---|---|---|---|---|
| Control | I collagen | CP 90 | CP45 | CP 20 | CP 16 | CP 9 |
| 1 | 0.83 | 0.79 | 0.83 | 0.88 | 0.79 | 0.99 |

In FIG. 8 and table 8 the mean values (n=6) based on untreated control cells with regard to IFNγ are presented. Unless otherwise stated, each measured value was statistically significantly (p<0.05) different from the controls. n.s.=not significant based on the control group.

TABLE 8

| | Native type | | | | | |
|---|---|---|---|---|---|---|
| Control | I collagen | CP 90 | CP45 | CP 20 | CP 16 | CP 9 |
| 1 | 0.92 | 0.89 | 0.87 | 0.93 | 0.90 | 1 |

The reduced synthesis rates of 0.56-fold for TNFα (FIG. 5), 0.75-fold for IL-1β (FIG. 6), 0.79-fold for IL-6 (FIG. 7) and 0.87-fold for IFNγ (FIG. 8) determined in the immunosuppressive T-suppressor cells showed a clear, statistically significant, advantageous effect of the tested collagen peptides compared to the untreated control. After application of CP9, however, there was no corresponding positive effect compared to the control.

The data determined show a clear, statistically significant, advantageous effect of the tested type I collagen peptides (CP90, CP45, CP20 and CP16) on the formation of pro-inflammatory cytokines in immunosuppressive T-suppressor cells whose differentiation had been induced by the M2 macrophages from peripheral blood monocytes.

### EXAMPLE 4: PROOF OF CONCEPT—IMMUNOSUPPRESSION IN DERMAL CONNECTIVE TISSUE CELLS

Human dermal fibroblasts were cultivated in Hams-F12 medium (HAM-12-A, Capricorn, Germany) supplemented with 1% amphotericin and with 1% penicillin-streptomycin and 10% calf serum. After reaching 100 percent cell confluence, an inflammatory situation was induced in the fibroblasts by adding 1 μg/ml lipopolysaccharide (Escherichia coli, L6529, Merck, Germany). The addition of 25 μl/ml cell supernatant from the T-cell differentiation experiment (example 3) reduced the inflammation in the dermal fibroblasts.

The studies were carried out on dermal fibroblasts. In FIG. 9 and table 9 the mean values (n=6) based on untreated control cells with regard to IL-1β are presented. Unless otherwise stated, each measured value was statistically significantly (p<0.05) different from the controls. n.s.=not significant based on the control group.

TABLE 9

| | Native type | | | | | |
|---|---|---|---|---|---|---|
| Control | I collagen | CP 90 | CP45 | CP 20 | CP 16 | CP 9 |
| 1 | 0.34 | 0.20 | 0.25 | 0.31 | 0.38 | 0.88 |

In FIG. 10 and table 10 the mean values (n=6) based on untreated control cells with regard to TNF-α are presented. Unless otherwise stated, each measured value was statistically significantly (p<0.05) different from the controls. n.s.=not significant based on the control group.

TABLE 10

| | Native type | | | | | |
|---|---|---|---|---|---|---|
| Control | I collagen | CP 90 | CP45 | CP 20 | CP 16 | CP 9 |
| 1 | 0.88 | 0.90 | 0.85 | 0.88 | 0.89 | 0.99 |

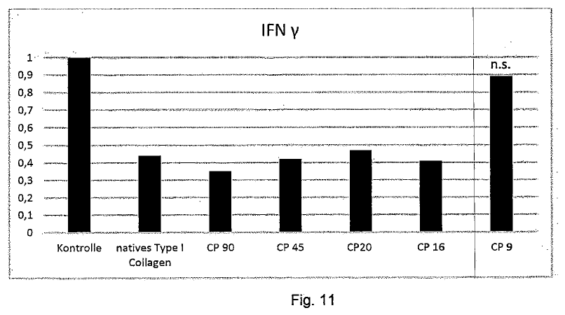

In FIG. 11 and Table 11 the mean values (n=6) based on untreated control cells with regard to IFNγ are presented. Unless otherwise stated, each measured value was statistically significantly (p<0.05) different from the controls. n.s.=not significant based on the control group.

TABLE 11

| | Native type | | | | | |
|---|---|---|---|---|---|---|
| Control | I collagen | CP 90 | CP45 | CP 20 | CP 16 | CP 9 |
| 1 | 0.44 | 0.35 | 0.42 | 0.47 | 0.41 | 0.89 |

An anti-inflammatory effect of the tested collagen peptides and native type I collagen in dermal fibroblasts was demonstrated by the up to 0.2-fold reduction in the expression of IL-1β (FIG. 5), the up to 0.85-fold reduction in the expression of TNFα (FIG. 6) and the maximum 0.35-fold reduction in the expression of IFNγ (FIG. 7). In contrast, the CP9 peptide showed no statistically significant anti-inflammatory effect.

Finally, based on a reduced expression of pro-inflammatory cytokines (IL-1β, TNFα and IFNγ) in the fibroblasts, the anti-inflammatory effect of CP90, CP45, CP20 and CP16 in dermal fibroblasts of the dermal target tissue was detected by real-time PCR.

1. A therapeutic method for oral therapy of inflammatory skin or a mucous membrane disease, in particular intestinal mucous membrane diseases, of a human or animal patient, comprising administering to the patient a type I collagen, wherein the collagen has a molecular weight of at least 16 kDa.

2. The method of claim 1, wherein the inflammatory skin and mucous membrane disease is an immunomodulated inflammatory skin and mucous membrane disease, in particular an autoimmune disease, in particular of the skin, such as cutaneous lupus erythematosus, dermatomyositis, lichen sclerosus, neurodermatitis, psoriasis, rosacea, lichen ruber, bullous pemphigoid, pemphigus vulgaris and acne or of the mucous membrane, in particular the intestinal mucous membrane, such as Crohn's disease, coeliac disease or ulcerative colitis.

3. The method of claim 1, wherein the recombinant or natural type I collagen, in particular the type I collagen peptide, with a molecular weight of at least 16 kDa is free of collagen, in particular also free of collagen peptides, with a molecular weight of less than 16 kDa.

4. The method of claim 1, wherein the type I collagen is present in triple-helical form.

5. The method according to claim 1, wherein the type I collagen is present in the form of a natural or recombinant type I collagen, in particular type I collagen peptide, preferably bovine type I collagen peptide comprising the amino acid sequence according to SEQ ID No. 2, 4, 6 or 8.

6. The method of claim 1, wherein the recombinant or natural type I collagen, in particular collagen peptide, has a molecular weight in a range of 35 to 95, in particular 40 to 92 kDa.

7. The method of claim 1, wherein the recombinant or natural type I collagen is denatured or non-denatured.

8. The method of claim 1, wherein the type I collagen is present fully or partially hydroxylated, fully or partially glycosylated or fully or partially hydroxylated and glycosylated.

9. The method of claim 1, wherein the recombinant type I collagen has been prepared by expression in a eukaryotic host cell, in particular a yeast cell, or prokaryotic host cell, in particular E. coli, in particular in hydroxylated form and/or in the form of a fusion peptide.

10. The method of claim 1, wherein the recombinant or natural type I collagen, in particular recombinant or natural type I collagen peptide, is present in isolated homogeneous form with a uniform molecular weight in a range of at least 16 kDa or as an isolated type I collagen mixture, in particular type I collagen peptide mixture, wherein all type I collagens have a molecular weight of at least 16 kDa.

11. The method of claim 1, wherein the type I collagen is the type I collagen of a vertebrate, in particular pig, sheep, cattle, rodent, kangaroo, horse, bird, reptile, amphibian, or fish, or of an invertebrate, in particular jellyfish.

12. A therapeutic method for oral therapy of inflammatory skin or a mucous membrane disease, in particular intestinal mucous membrane diseases in a subject, comprising administering to the subject a composition comprising at least one type I collagen with a molecular weight of at least 16 kDa of claim 1 and at least one pharmaceutically acceptable or food safe carrier and, optionally, at least one additive or excipient.

13. The method of claim 12, wherein the at least one excipient is a corticosteroid.

14. The method of claim 12, wherein the at least one additive is a recombinantly produced collagen hydrolysate, a collagen hydrolysate derived from natural sources, a recombinantly produced type I collagen, a type I collagen recovered from a natural source, or a combination thereof.

15. The method of claim 12, wherein the composition is present in form of a tablet, lozenge, chewable tablet, powder, granules, hard capsule, soft capsule, capsule, bite capsule, dragee, pastille, extrudate, juice, suspension or gel.

16. The method of claim 12, wherein the composition is suitable for oral administration in a dose of 1 to 60 mg/day of type I collagen.

17. A therapeutic treatment or therapeutic prophylaxis, in a subject, of immune intolerance to type I collagen, in particular in immunomodulated inflammatory skin and mucous membrane diseases, in particular intestinal mucous membrane diseases, comprising administering to the subject a type I collage, in particular a recombinant or natural type I collagen peptide, with a molecular weight of at least 16 kDa.

18. A method for inducing immune tolerance to type I collagen in a subject, in particular in immunomodulated inflammatory skin and mucous membrane diseases, in particular intestinal mucosal membrane diseases, comprising administering to the subject a type I collage, in particular a recombinant or natural type I collagen peptide, with a molecular weight of at least 16 kDa.

19. (canceled)


SEQUENCE LISTING (9 sequences)

SEQ ID NO: 1 - DNA, 547 bp, synthetic construct
  misc_feature 1..547: note = CP 16
  source 1..547: mol_type = other DNA
gagagaggtttccccggcgagcgtggtgtgcaagggccgcccggtcctgcaggtccccgtggggccaatggtgcccctggcaacgatggtgctaagggtgatgctggtgcccctggagcccccggtagccagggtgcccctggccttcaaggaatgcctggtgaacgaggtgcagctggtcttccaggccctaagggtgacagaggggatgctggtcccaaaggtgctgatggtgctcctggcaaagatggcgtccgtggtctgactggtcccatcggtcctcctggccccgctggtgcccctggtgacaagggtgaagctggtcctagtggcccagccggtcccactggagctcgtggtgcccccggtgaccgtggtgagcctggtccccccggccctgctggcttcgctggcccccctggtgctgatggccaacctggtgctaaaggcgaacctggtgatgctggtgctaaaggtgacgctggtccccccggccctgctgggcccgctggaccccccggccccattggtaacgttggtgctcccg

SEQ ID NO: 2 - protein, 182 aa, synthetic construct
  REGION 1..182: note = CP 16
ERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAP

SEQ ID NO: 3 - DNA, 694 bp, synthetic construct
  misc_feature 1..694: note = CP 20
  source 1..694: mol_type = other DNA
gagagaggtttccccggcgagcgtggtgtgcaagggccgcccggtcctgcaggtccccgtggggccaatggtgcccctggcaacgatggtgctaagggtgatgctggtgcccctggagcccccggtagccagggtgcccctggccttcaaggaatgcctggtgaacgaggtgcagctggtcttccaggccctaagggtgacagaggggatgctggtcccaaaggtgctgatggtgctcctggcaaagatggcgtccgtggtctgactggtcccatcggtcctcctggccccgctggtgcccctggtgacaagggtgaagctggtcctagtggcccagccggtcccactggagctcgtggtgcccccggtgaccgtggtgagcctggtccccccggccctgctggcttcgctggcccccctggtgctgatggccaacctggtgctaaaggcgaacctggtgatgctggtgctaaaggtgacgctggtccccccggccctgctgggcccgctggaccccccggccccattggtaacgttggtgctcccggacccaaaggtgctcgtggcagcgctggtccccctggtgctactggtttcccaggtgctgctggccgagtcggtccccccggcccctctggaaatgctggaccccctggccctcctggccctgctggcaaagaaggcagcaaaggcc

SEQ ID NO: 4 - protein, 231 aa, synthetic construct
  REGION 1..231: note = CP 20
ERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKG

SEQ ID NO: 5 - DNA, 1548 bp, synthetic construct
  misc_feature 1..1548: note = CP 45
  source 1..1548: mol_type = other DNA
gagagaggtttccccggcgagcgtggtgtgcaagggccgcccggtcctgcaggtccccgtggggccaatggtgcccctggcaacgatggtgctaagggtgatgctggtgcccctggagcccccggtagccagggtgcccctggccttcaaggaatgcctggtgaacgaggtgcagctggtcttccaggccctaagggtgacagaggggatgctggtcccaaaggtgctgatggtgctcctggcaaagatggcgtccgtggtctgactggtcccatcggtcctcctggccccgctggtgcccctggtgacaagggtgaagctggtcctagtggcccagccggtcccactggagctcgtggtgcccccggtgaccgtggtgagcctggtccccccggccctgctggcttcgctggcccccctggtgctgatggccaacctggtgctaaaggcgaacctggtgatgctggtgctaaaggtgacgctggtccccccggccctgctgggcccgctggaccccccggccccattggtaacgttggtgctcccggacccaaaggtgctcgtggcagcgctggtccccctggtgctactggtttcccaggtgctgctggccgagtcggtccccccggcccctctggaaatgctggaccccctggccctcctggccctgctggcaaagaaggcagcaaaggcccccgcggtgagactggccccgctgggcgtcccggtgaagtcggtccccctggtccccctggccccgctggtgagaaaggagcccctggtgctgacggacctgctggagctcctggcactcctggacctcaaggtattgctggacagcgtggtgtggtcggcctgcctggtcagagaggagaaagaggcttccctggtcttcctggcccctctggtgaacccggcaaacaaggtccttctggagcaagtggtgaacgtggcccccctggtcccatgggcccccctggattggctggaccccctggcgagtctggacgtgagggagctcctggtgctgaaggatcccctggacgagatggttctcctggcgccaagggtgaccgtggtgagaccggccctgctggacctcctggtgctcctggcgctcccggtgcccccggccctgtcggacctgccggcaagagcggtgatcgtggtgagaccggtcctgctggtcctgctggtcccattggccccgttggtgcccgtggccccgctggaccccaaggcccccgtggtgacaagggtgagacaggcgaacagggcgacagaggcattaagggtcaccgtggcttctctggtctccagggtccccccggccctcccggctctcctggtgagcaaggtccttccggagcctctggtcctgctggtccccgcggtccccctggctctgctggttctcccggcaaagatggactcaatggtctcccaggccccatcggtccccctgggcctcgaggtcgcactggtgatgctggtcctgctggtcctcccggccctcctggaccccctggtcccccaggtcctcccagc

SEQ ID NO: 6 - protein, 516 aa, synthetic construct
  REGION 1..516: note = CP 45
ERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPS

SEQ ID NO: 7 - DNA, 3093 bp, synthetic construct
  misc_feature 1..3093: note = CP 90
  source 1..3093: mol_type = other DNA
cagttgtcttacggctatgatgagaaatcaacaggaatttccgtgcctggtcccatgggtccttctggtcctcgtggtctccctggcccccctggcgcacctggtccccaaggtttccaaggcccccctggtgagcctggcgagccaggagcctcaggtcccatgggtccccgtggtccccctggcccccctggcaagaacggagatgatggcgaagctggaaagcctggtcgtcctggtgagcgcgggcctcccggacctcagggtgctcggggattgcctggaacagctggcctccctggaatgaagggacacagaggtttcagtggtttggatggtgccaagggagatgctggtcctgctggccccaagggcgagcctggtagccccggtgaaaatggagctcctggtcagatgggcccccgtggtctgcctggtgagagaggtcgccctggagcccctggccctgctggtgctcgaggaaatgatggtgcgactggtgctgctgggccccctggtcccactggccccgctggtcctcctggtttccctggtgctgtgggtgctaagggtgaaggtggtccccaaggaccccgaggttctgaaggtccccagggtgtacgtggtgagcctggcccccctggccctgctggtgctgctggccctgctggcaaccctggtgctgatggacagcctggtgctaaaggagccaatggcgctcctggtattgctggtgctcctggcttccctggtgcccgaggcccctctggaccccagggccccagcggcccccctggccccaagggtaacagcggtgaacctggtgctcctggcagcaaaggagacactggcgccaagggagaacccggtcccactggtattcaaggcccccctggccccgctggggaagaaggaaagcgaggagcccgaggtgaacctggacctgctggcctgcctggaccccctggcgagcgtggtggacctggaagccgtggtttccctggcgccgacggtgttgctggtcccaagggtcctgctggtgaacgcggtgctcctggccctgctggccccaaaggttctcctggtgaagctggtcgccccggtgaagctggtctgcccggtgccaagggtctgactggaagccctggcagcccgggtcctgatggcaaaactggcccccctggtcccgccggtcaagatggccgccctggacctccaggccctcccggtgcccgtggtcaggctggcgtgatgggtttccctggacctaaaggtgctgctggagagcctggaaaagctggagagcgaggtgttcctggaccccctggcgctgttggtcctgctggcaaagacggagaagctggagctcagggacccccaggacctgctggccccgctggtgagagaggcgaacaaggccctgctggctcccctggattccagggtctccccggccctgctggtcctcctggtgaagcaggcaaacctggtgaacagggtgttcctggagatcttggtgcccccggcccctctggagcaagaggcgagagaggtttccccggcgagcgtggtgtgcaagggccgcccggtcctgcaggtccccgtggggccaatggtgcccctggcaacgatggtgctaagggtgatgctggtgcccctggagcccccggtagccagggtgcccctggccttcaaggaatgcctggtgaacgaggtgcagctggtcttccaggccctaagggtgacagaggggatgctggtcccaaaggtgctgatggtgctcctggcaaagatggcgtccgtggtctgactggtcccatcggtcctcctggccccgctggtgcccctggtgacaagggtgaagctggtcctagtggcccagccggtcccactggagctcgtggtgcccccggtgaccgtggtgagcctggtccccccggccctgctggcttcgctggcccccctggtgctgatggccaacctggtgctaaaggcgaacctggtgatgctggtgctaaaggtgacgctggtccccccggccctgctgggcccgctggaccccccggccccattggtaacgttggtgctcccggacccaaaggtgctcgtggcagcgctggtccccctggtgctactggtttcccaggtgctgctggccgagtcggtccccccggcccctctggaaatgctggaccccctggccctcctggccctgctggcaaagaaggcagcaaaggcccccgcggtgagactggccccgctgggcgtcccggtgaagtcggtccccctggtccccctggccccgctggtgagaaaggagcccctggtgctgacggacctgctggagctcctggcactcctggacctcaaggtattgctggacagcgtggtgtggtcggcctgcctggtcagagaggagaaagaggcttccctggtcttcctggcccctctggtgaacccggcaaacaaggtccttctggagcaagtggtgaacgtggcccccctggtcccatgggcccccctggattggctggaccccctggcgagtctggacgtgagggagctcctggtgctgaaggatcccctggacgagatggttctcctggcgccaagggtgaccgtggtgagaccggccctgctggacctcctggtgctcctggcgctcccggtgcccccggccctgtcggacctgccggcaagagcggtgatcgtggtgagaccggtcctgctggtcctgctggtcccattggccccgttggtgcccgtggccccgctggaccccaaggcccccgtggtgacaagggtgagacaggcgaacagggcgacagaggcattaagggtcaccgtggcttctctggtctccagggtccccccggccctcccggctctcctggtgagcaaggtccttccggagcctctggtcctgctggtccccgcggtccccctggctctgctggttctcccggcaaagatggactcaatggtctcccaggccccatcggtccccctgggcctcgaggtcgcactggtgatgctggtcctgctggtcctcccggccctcctggaccccctggtcccccaggtcctcccagc

SEQ ID NO: 8 - protein, 1031 aa, synthetic construct
  REGION 1..1031: note = CP 90
QLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPS

SEQ ID NO: 9 - protein, 90 aa, synthetic construct
  REGION 1..90: note = Artificial collagen sequence
  MOD_RES 3: note = 4Hyp
  MOD_RES 18: note = 4Hyp
  MOD_RES 24: note = 4Hyp
  MOD_RES 45: note = 4Hyp
  MOD_RES 51: note = 4Hyp
  MOD_RES 54: note = 4Hyp
  MOD_RES 63: note = 4Hyp
  MOD_RES 69: note = 4Hyp
GAPGKDGVRGLTGPIGPPGPAGAPGDKGEAGPSGPAGPTGARGAPGDRGEPGPPGPAGFAGPPGADGQPGAKGEPGDAGAKGDAGPPGPA